(19) United States
(12) Patent Application Publication
(10) Pub. No.: US 2022/0163519 A1
(43) Pub. Date: May 26, 2022

(54) BIOMIMETIC NANOVILLI CHIPS FOR ENHANCED CAPTURE OF TUMOR-DERIVED EXTRACELLULAR VESICLES

(71) Applicant: The Regents of the University of California, Oakland, CA (US)

(72) Inventors: Hsian-Rong Tseng, Los Angeles, CA (US); Yazhen Zhu, Los Angeles, CA (US); Jiantong Dong, Los Angeles, CA (US)

(73) Assignee: The Regents of the University of California, Oakland, CA (US)

(21) Appl. No.: 17/440,653

(22) PCT Filed: Mar. 19, 2020
(86) PCT No.: PCT/US2020/023656

§ 371 Date: Sep. 17, 2021

Related U.S. Application Data
(60) Provisional application No. 62/821,026, filed on Mar. 20, 2019.

(51) Int. Cl. G01N 33/543; B01L 3/00

(52) U.S. Cl. CPC G01N 33/54366; B01L 3/502761; B01L 2300/08; B01L 2200/16; B01L 2200/0652

(57) ABSTRACT

Methods and kits for capturing extracellular vesicles from a fluid sample, including: providing a microfluidic chip having a device for capturing extracellular vesicles from the fluid sample; flowing the fluid sample through a fluid channel defined by a channel-defining layer in the microfluidic chip so as to capture extracellular vesicles from the fluid sample; removing a membrane from the device for capturing extracellular vesicles after providing the fluid sample; and collecting the extracellular vesicles captured from the fluid sample.

### CROSS-REFERENCE OF RELATED APPLICATIONS

This application claims priority to U.S. Provisional Application No. 62/821,026 filed Mar. 20, 2019; the entire contents of which are hereby incorporated by reference.

### GOVERNMENT SUPPORT CLAUSE

This invention was made with government support under Grant Number CA198900, awarded by the National Institutes of Health. The government has certain rights in the invention.

### BACKGROUND

### 1. Technical Field

The field of the currently claimed embodiments of this invention relate to methods and systems for assessing a disease condition of a cancer of a subject by isolating and assaying circulating extracellular vesicles.

### 2. Discussion of Related Art

Extracellular vesicles' (EVs) are a heterogeneous group of lipid bilayer-enclosed particles that play a crucial role in intercellular communication by transporting biomolecular cargo, including DNA, RNA, proteins, and lipids.^{2,3} Extracellular vesicles are classified into three categories according to their size and their biogenesis pathway of origin: i) exosomes (30-150 nm);^{4,5} ii) microvesicles (100-1000 nm);⁶ iii) apoptotic bodies (500-4000 nm).⁷ Extracellular vesicles are actively secreted by all cell types in the human body and can be found in a variety of body fluids. Oncogenic transformation often leads to increased EV production by tumor cells, resulting in increased levels of tumor-derived EVs in patients' blood.^{8,9} Compared to well-studied circulating tumor cells (CTCs), which are challenging to detect until metastatic progression, tumor-derived EVs are present in circulation at relatively early stages of disease. These cancer-specific EVs can be collected from plasma or serum at any time over the course of treatment. Consequently, tumor-derived EVs are emerging candidates for liquid biopsy approaches¹⁰⁻¹² for implementing non-invasive cancer diagnosis, prognosis, and treatment monitoring across all disease stages.

Since the biomolecular contents of tumor-derived EVs mirror those of the parental tumor cells, performing molecular characterization on tumor-derived EVs could provide key insights into the molecular mechanisms governing oncogenesis and disease progression. Most importantly, the fragile biomolecular contents inside individual EVs (e.g., tumor-specific RNA) are protected by the EV's lipid bilayer, guaranteeing their availability for downstream molecular analysis. Recent studies have demonstrated the feasibility of detecting cancer driver mutations using mRNA extracted from enriched tumor-derived EVs in different solid tumors, for example, KRAS mutations in pancreatic cancer¹³ and EGFR vIII mutation in glioblastoma.¹⁴ Performing mutational analyses using EV-derived mRNA results in improved sensitivity and better correlation with patients' clinical outcomes over cell-free DNA (cfDNA)-based approaches.^{15,16} Moreover, well-preserved RNA in tumor-derived EVs is ideal for detecting gene rearrangements, as they have variable breakpoints and different fusion partners.

Since tumor-derived EVs constitute only a minor portion of the total number of EVs in circulation, the enrichment of tumor-derived EVs represents a considerable technical challenge. Conventional methods such as ultracentrifugation,¹⁷⁻²⁰ filtration,^{21,22} precipitation,²³ size-based microfluidic enrichment,²⁴⁻²⁹ can isolate entire populations of EVs from peripheral blood samples based on their physical properties (i.e., size and/or density). However, these approaches are incapable of discriminating tumor-derived EVs from non-tumor-derived EVs. More recent research efforts have explored the application of immunoaffinity-based capture techniques for enriching tumor-derived EVs in different solid tumors.^{17,18,27} For example, pancreatic cancer-derived exosomes can be captured selectively using anti-GPC1-coated beads and isolated via flow cytometry,¹³ and the enrichment of glioblastoma-derived exosomes has been demonstrated in herringbone microfluidic devices (i.e., ^{EV}HB-Chip) with EGFRvIII antibodies used as the capture agent.¹⁴ To characterize and/or to quantify the trace amount of mRNA extracted from the enriched tumor-derived EVs, highly sensitive mRNA profiling technologies, e.g., next-generation sequencing and Droplet Digital™ PCR (ddPCR) were adopted for downstream detection purposes.

Therefore there remains a need for improved methods and systems for assessing a disease condition of a cancer of a subject by isolating and assaying circulating extracellular vesicles.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a method for capturing extracellular vesicles from a fluid sample including: providing a microfluidic chip, the microfluidic chip having: a device for capturing extracellular vesicles from a fluid sample having: a substrate; and a plurality of nanowires at least one of attached to or integral with a surface of the substrate such that each nanowire of said plurality of nanowires has an unattached end; and a membrane disposed on the device for capturing extracellular vesicles, the membrane having a fluid channel defined by a channel-defining layer. In such an embodiment, the membrane is removable from the device for capturing extracellular vesicles, the plurality of nanowires include a binding agent attached to a surface region of the plurality of nanowires, and the channel-defining layer defines the fluid channel such that at least a portion of the fluid channel has a chaotic mixing structure to cause at least partially turbulent flow. The method also includes flowing the fluid sample through the fluid channel defined by the channel-defining layer so as to capture extracellular vesicles from the fluid sample; removing the membrane from the device for capturing extracellular vesicles after the providing the fluid sample; and collecting the extracellular vesicles captured from the fluid sample.

An embodiment of the invention relates to a method of determining the presence of a cancer cell in a subject, including: providing a microfluidic chip for capturing extracellular vesicles from a fluid sample, the microfluidic chip having: a device for capturing extracellular vesicles from the fluid sample having: a substrate; and a plurality of nanowires at least one of attached to or integral with a surface of the substrate such that each nanowire of said plurality of nanowires has an unattached end; and a membrane disposed on the device for capturing extracellular vesicles, the membrane comprising a fluid channel defined by a channel-defining layer. In such an embodiment, the membrane is removable from the device for capturing extracellular vesicles, the plurality of nanowires comprise a binding agent attached to a surface region of the plurality of nanowires, and the channel-defining layer defines the fluid channel such that at least a portion of the fluid channel has a chaotic mixing structure to cause at least partially turbulent flow. The method also includes flowing the fluid sample through the fluid channel defined by the channel-defining layer so as to capture extracellular vesicles from the fluid sample; assaying the captured extracellular vesicles for a presence of a biomarker associated with the cancer cell.

An embodiment of the invention relates to a kit for capturing extracellular vesicles from a fluid sample having: a microfluidic system for capturing extracellular vesicles from a fluid sample having: a device for capturing extracellular vesicles from a fluid sample having: a substrate; and a plurality of nanowires at least one of attached to or integral with a surface of the substrate such that each nanowire of said plurality of nanowires has an unattached end; and a membrane disposed on the device for capturing extracellular vesicles, the membrane comprising a fluid channel defined by a channel-defining layer; a binding agent attached to a surface region of the plurality of nanowires; and reagents for assaying the captured extracellular vesicles for a presence of a biomarker. In such an embodiment, the membrane is removable from the device for capturing extracellular vesicles, and the channel-defining layer defines the fluid channel such that at least a portion of the fluid channel has a chaotic mixing structure to cause at least partially turbulent flow.

### BRIEF DESCRIPTION OF THE DRAWINGS

FIGS. 1A and 1B show an illustration showing the structures of intestinal microvilli and a schematic of a device and method according to an embodiment.

FIGS. 2A-2H are images, graphs, and schematics showing the characterization of tumor-derived extracellular vesicles (EVs) in solution and on anti-epithelial cell adhesion molecule (anti-EpCAM)-grafted silicon nanowire substrates (SiNWS) in NanoVilli Chips according to an embodiment.

FIGS. 3A-3F are graphs and images showing optimization of NanoVilli Chips for immunoaffinity capture of tumor-derived extracellular vesicles (EVs) using artificial plasma samples according to an embodiment.

FIGS. 4A-4D are images and graphs demonstrating that NanoVilli Chips combined with reverse transcription Droplet Digital™ PCR (RT-ddPCR) analysis can be used to detect and to monitor ROS1 rearrangements or acquired EGFR T790M mutation in tumor-derived EVs purified from non-small cell lung cancer (NSCLC) patients' blood according to an embodiment.

FIG. 5 is a scheme illustrating the surface chemical modification process used to prepare anti-epithelial cell adhesion molecule (anti-EpCAM)-grafted silicon nanowire substrates (SiNWS) according to an embodiment.

FIG. 6 is a photograph and a schematic showing the setup of the entire NanoVilli device according to an embodiment.

FIGS. 7A-7H are images and graphs showing tumor-derived extracellular vesicles (EVs) captured on anti-epithelial cell adhesion molecule (anti-EpCAM)-grafted silicon nanowire substrates (SiNWS) characterized using scanning electron microscopy (SEM) and transmission electron microscopy (TEM) according to an embodiment.

FIGS. 8A and 8B are scanning electron microscopy (SEM) images showing the different extracellular vesicle (EV) capture performance on a standard NanoVilli Chip (which is conjugated with anti-EpCam) and a control device (a NanoVilli Chip without antibody conjugation) according to an embodiment.

FIGS. 9A-9E are images and graphs showing extracellular vesicle (EV) distribution probability profiles along the depth of Si nanowires analyzed by scanning electron microscopy (SEM) and computational simulation according to an embodiment.

FIGS. 10A and 10B are graphs showing extracellular-vesicle-capture performance of NanoVilli Chips according to an embodiment.

FIGS. 11A-11D are schemes illustrating reverse transcription Droplet Digital™ PCR (RT-ddPCR) analysis of gene alterations from extracellular vesicle (EV)-derived RNA according to an embodiment.

FIGS. 12A-12D are images, graphs and schematics showing that leucine-rich repeat and Ig domain protein 1 (LINGO1) enables specific capture of and molecular analysis of Ewing sarcoma (EWS)-derived extracellular vesicles (EVs) according to an embodiment.

FIGS. 13A-13F are images, graphs and schematics showing the morphological characterization of A673 EVs captured via the reaction of TCO-anti-LINGO1 conjugates and Tz-grafted Si nanowires in Click^{EV} chips by electron microscopy according to an embodiment.

FIGS. 14A-14D are graphs showing validation and optimization of Click^{EV} Chips for LINGO1 induced capture of Ewing sarcoma (EWS) cell-derived EVs followed by quantification of EV-derived RNA according to an embodiment.

FIG. 15 is a scheme illustrating a nanostructured Click chip for specific recovery of tumor-derived EVs via multi-markers according to an embodiment.

FIGS. 16A-16F are graphs and schematics showing validation and optimization of Click Chips using artificial plasma samples spiked with 22RV1 cell-derived EVs according to an embodiment.

FIG. 17 is a scheme illustrating a nanostructured Click chip for specific recovery of HCC-derived EVs via multi-markers according to an embodiment.

FIGS. 18A-18F are images and graphs showing validation and optimization of Click Chips using artificial plasma samples spiked with HepG2 cell-derived EVs according to an embodiment.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the present invention are directed to a method for capturing extracellular vesicles from a fluid sample including: providing a microfluidic chip, the microfluidic chip having: a device for capturing extracellular vesicles from a fluid sample including: a substrate; and a plurality of nanowires at least one of attached to or integral with a surface of the substrate such that each nanowire of the plurality of nanowires has an unattached end; and a membrane disposed on the device for capturing extracellular vesicles, the membrane including a fluid channel defined by a channel-defining layer. IN such an embodiment, the membrane is removable from the device for capturing extracellular vesicles, the plurality of nanowires include a binding agent attached to a surface region of the plurality of nanowires, and the channel-defining layer defines the fluid channel such that at least a portion of the fluid channel has a chaotic mixing structure to cause at least partially turbulent flow. The method also includes flowing the fluid sample through the fluid channel defined by the channel-defining layer so as to capture extracellular vesicles from the fluid sample; removing the membrane from the device for capturing extracellular vesicles after the providing the fluid sample; and collecting the extracellular vesicles captured from the fluid sample.

Some embodiments of the present invention are directed to the method above where the binding agent has a plurality of antibodies, and the plurality of antibodies bind to two or more distinct targets.

Some embodiments of the present invention are directed to the method above where each of the plurality of nanowires has a length between 3-15 micrometers.

Some embodiments of the present invention are directed to the method above where each of the plurality of nanowires has a length between 10-15 micrometers.

Some embodiments of the present invention are directed to the method above where the chaotic mixing structure is configured in a herringbone pattern.

Some embodiments of the present invention are directed a method of determining the presence of a cancer cell in a subject, including: providing a microfluidic chip for capturing extracellular vesicles from a fluid sample, the microfluidic chip having: a device for capturing extracellular vesicles from the fluid sample having: a substrate; and a plurality of nanowires at least one of attached to or integral with a surface of the substrate such that each nanowire of the plurality of nanowires has an unattached end; and a membrane disposed on the device for capturing extracellular vesicles, the membrane including a fluid channel defined by a channel-defining layer. In such an embodiment, the membrane is removable from the device for capturing extracellular vesicles, the plurality of nanowires include a binding agent attached to a surface region of the plurality of nanowires, and the channel-defining layer defines the fluid channel such that at least a portion of the fluid channel has a chaotic mixing structure to cause at least partially turbulent flow. The method also includes flowing the fluid sample through the fluid channel defined by the channel-defining layer so as to capture extracellular vesicles from the fluid sample; assaying the captured extracellular vesicles for a presence of a biomarker associated with the cancer cell.

Some embodiments of the present invention are directed to the method further including obtaining the fluid sample from the subject.

Some embodiments of the present invention are directed to the method above where the binding agent includes a plurality of antibodies, and wherein the plurality of antibodies bind to two or more distinct targets.

Some embodiments of the present invention are directed to the method above where each of the plurality of nanowires has a length between 3-15 micrometers.

Some embodiments of the present invention are directed to the method above where each of the plurality of nanowires has a length between 10-15 micrometers.

Some embodiments of the present invention are directed to the method above where the chaotic mixing structure is configured in a herringbone pattern.

Some embodiments of the present invention are directed to the method above where the biomarker is a protein or a nucleic acid sequence.

Some embodiments of the present invention are directed to a kit for capturing extracellular vesicles from a fluid sample having: a microfluidic system for capturing extracellular vesicles from a fluid sample having: a device for capturing extracellular vesicles from a fluid sample having: a substrate; and a plurality of nanowires at least one of attached to or integral with a surface of the substrate such that each nanowire of the plurality of nanowires has an unattached end; and a membrane disposed on the device for capturing extracellular vesicles, the membrane including a fluid channel defined by a channel-defining layer; a binding agent attached to a surface region of the plurality of nanowires; and reagents for assaying the captured extracellular vesicles for a presence of a biomarker. In such an embodiment, the membrane is removable from the device for capturing extracellular vesicles, and the channel-defining layer defines the fluid channel such that at least a portion of the fluid channel has a chaotic mixing structure to cause at least partially turbulent flow.

Some embodiments of the present invention are directed to the kit above where the binding agent includes a plurality of antibodies, and the plurality of antibodies bind to two or more distinct targets.

Some embodiments of the present invention are directed to the kit above where each of the plurality of nanowires has a length between 3-15 micrometers.

Some embodiments of the present invention are directed to the kit above where each of the plurality of nanowires has a length between 10-15 micrometers.

Some embodiments of the present invention are directed to the kit above where the chaotic mixing structure is configured in a herringbone pattern.

In some embodiments directed to methods and systems for assessing a disease condition of a cancer of a subject, a NanoVelcro assay is used, by which anti-EpCAM (epithelial cell adhesion molecule)-coated nanostructured substrates (e.g., vertically oriented silicon nanowire substrates, SiNWS) are utilized to capture CTCs in a stationary device setting with a capture efficiency ranging from 40 to 70%. (See, for example, U.S. Pat. No. 9,140,697, “Device for Capturing Circulating Tumor Cells,” assigned to the same assignee as the current application, the entire contents of which are incorporated herein by reference.)

### 1. Definitions

To facilitate an understanding of the present invention, a number of terms and phrases are defined below.

The term “nanostructure” refers to a structure having a lateral dimension and a longitudinal dimension, wherein the lateral dimension, the longitudinal dimension, or both the lateral and longitudinal dimensions are less than 1 mm. The shape of the nanostructure is not critical. It can, for example, be any three dimensional structure such as, but not limited to, a bead, particle, strand, tube, sphere, etc.

The terms “diagnostic” and “diagnosis” refer to identifying the presence or nature of a pathologic condition and includes identifying patients who are at risk of developing a specific disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “detection”, “detecting” and the like, may be used in the context of detecting biomarkers, or of detecting a disease or disorder (e.g., when positive assay results are obtained). In the latter context, “detecting” and “diagnosing” are considered synonymous.

The terms “subject”, “patient” or “individual” generally refer to a human, although the methods of the invention are not limited to humans, and should be useful in other mammals (e.g., cats, dogs, etc.).

“Sample” is used herein in its broadest sense. A sample may include a bodily fluid including blood, serum, plasma, tears, aqueous and vitreous humor, spinal fluid, urine, and saliva; a soluble fraction of a cell or tissue preparation, or media in which cells were grown. Means of obtaining suitable biological samples are known to those of skill in the art.

The term “binding agent” as used herein refers to any entity or substance, e.g., molecule, which is associated with (e.g., immobilized on, or attached either covalently or non-covalently to) the nanostructured surface region, or which is a portion of such surface (e.g., derivatized portion of a plastic surface), and which can undergo specific interaction or association with the target cell. A “plurality of binding agents” can refer to a plurality of one particular binding agent or a plurality of more than one binding agent.

An “antibody” is an immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies generated from at least two intact antibodies, hybrid antibodies, fusion proteins including an antibody portion, and any other modified immunoglobulin molecule including an antigen recognition site so long as the antibodies exhibit the desired biological activity. An antibody may be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies may be naked or conjugated to other molecules such as toxins, radioisotopes, etc.

The term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments.

“Hybrid antibodies” are immunoglobulin molecules in which pairs of heavy and light chains from antibodies with different antigenic determinant regions are assembled together so that two different epitopes or two different antigens may be recognized and bound by the resulting tetramer.

“Isolated” in regard to cells or extracellular vesicles, refers to a cell or extracellular vesicle that is removed from its natural environment (such as in a solid tumor) and that is isolated or separated, and is at least about 30%, 50%, 75%, and 90% free from other cells with which it is naturally present, but which lack the marker based on which the cells were isolated.

That a molecule (e.g., binding agent) “specifically binds” to or shows “specific binding” or “captures” or “selectively captures” a target cell means that the molecule reacts or associates more frequently, more rapidly, with greater duration, and/or with greater affinity with the target cell than with alternative substances. Thus, under designated experimental conditions, the specified molecule bind to the target cell at least two times the background and does not substantially bind in a significant amount to other cells and proteins present in the sample.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The following describes some embodiments of the current invention more specifically. The general concepts of this invention are not limited to these particular embodiments.

### EXAMPLES

### Example 1

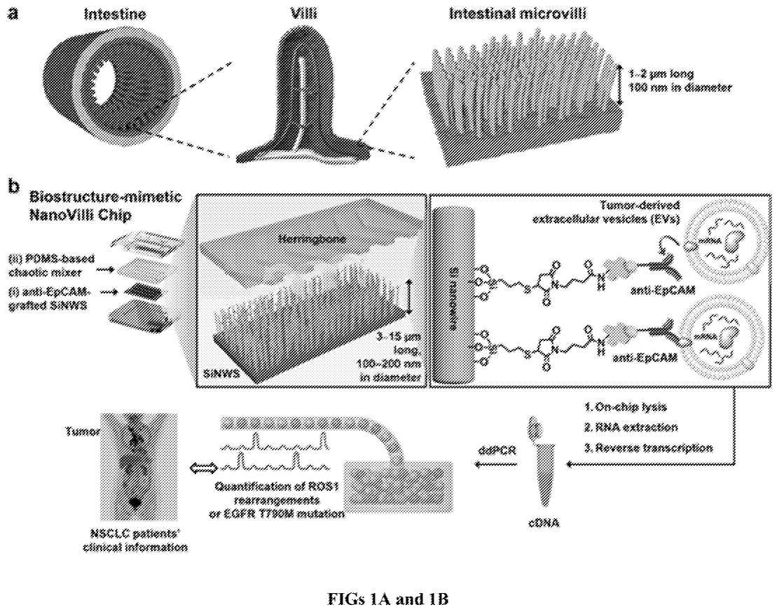

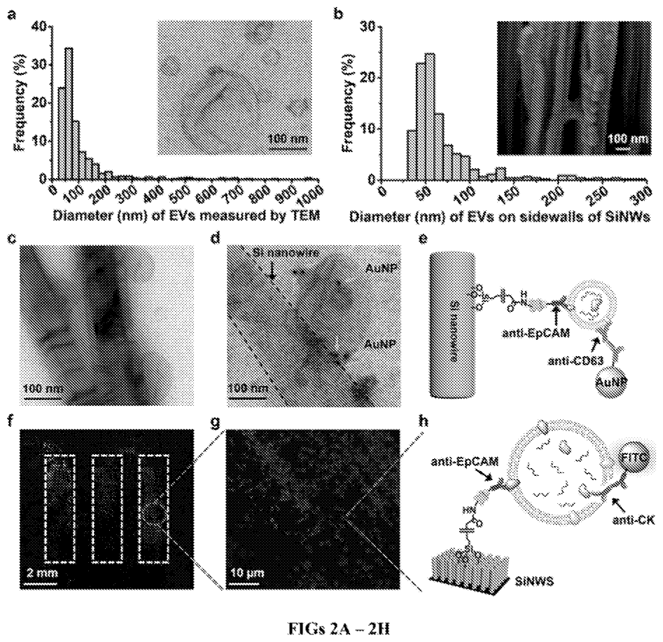

Inspired by the distinctive structures of intestinal microvilli (FIG. 1A), which are densely packed on intestinal walls to increase mucosal surface area for enhanced absorption, NanoVilli Chips were developed. These biostructure-inspired chips have antibody-grafted silicon (Si) nanowire arrays that are engineered in a densely packed manner to achieve efficient and reproducible immunoaffinity capture of tumor-derived EVs from blood plasma samples. A NanoVilli Chip is composed of two integral components (FIG. 1B), i.e., (i) an anti-epithelial cell adhesion molecule (EpCAM)-grafted Si nanowire substrate (SiNWS) and (ii) a superimposed polydimethylsiloxane (PDMS)-based chaotic mixer with a serpentine microchannel, in which herringbone micropatterns introduce helical flow to facilitate direct physical contact between anti-EpCAM-grafted SiNWS and tumor-derived EVs in plasma. When a plasma sample containing tumor-derived EVs is run through a NanoVilli Chip, the integration of the anti-EpCAM-grafted SiNWS and the PDMS-based chaotic mixer leads to enhanced capture of tumor-derived EVs. SiNWS are optimized for interacting with nanoscale targets. In previously described NanoVelcro chips,³⁰⁻³² which utilize a similar device configuration, Velcro-like topographic interactions between nanostructured substrates and nanoscale cellular surface components immobilize CTCs on top of the SiNWS. Whereas microscale CTCs (diameters=6-20 μm) can only interact with the uppermost portion of the SiNWS, free-floating nanoscale EVs (diameters=30-1000 nm) can interact with both upper and deeper portions of the SiNWS (which are spaced 200-400 nm apart). Extracellular vesicles above 300 nm in diameter are primarily captured on the tips of the SiNWS, whereas EVs with sizes ranging between 30 and 300 nm are captured on both tips and sidewalls of the SiNWS. NanoVilli Chips with longer Si nanowires (lengths=10-15 μm) were designed to increase functional surface area, enabling more efficient enrichment of tumor-derived EVs at both the tips and the sidewalls of individual Si nanowires. After capturing tumor-derived EVs on NanoVilli Chips, RNA recovered from the EVs can be evaluated with a Qubit™ 3.0 Fluorometer in combination with the Qubit RNA HS Assay and subjected to downstream analysis by reverse transcription Droplet Digital™ PCR (RT-ddPCR). The clinical utility of NanoVilli Chips was explored by applying this workflow to detect driver gene alterations in non-small cell lung cancer (NSCLC) quantitatively (e.g., ROS1 rearrangements or epidermal growth factor receptor, EGFR, T790M mutation). To understand how the embedded Si nanowires in NanoVilli Chips contribute to the highly efficient capture of tumor-derived EVs, scanning electron microscopy (SEM), transmission electron microscopy (TEM), and fluorescence microscopy were employed (FIGS. 2A-2H) to characterize the interactions between anti-EpCAM-grafted SiNWS and tumor-derived EVs. These observations were further validated with those obtained via computational simulation. Moreover, the performance of NanoVilli Chips was optimized by systematically varying device operating conditions and configurations (e.g., flow rates, Si nanowire lengths, and anti-EpCAM concentrations). These data were evaluated to identify experimental conditions that enable efficient and reproducible enrichment of tumor-derived EVs from both artificial plasma samples and blood plasma samples obtained from NSCLC patients. The combined use of NanoVilli Chips and RT-ddPCR offers a new type of EV-based mRNA assay for quantitatively detecting and monitoring targetable oncogenic gene alterations in NSCLC patients.

There has recently been a major strategic shift in the clinical management of NSCLC. Following initial tissue-based histological classification schemes, NSCLC has been further classified based on molecular phenotype (e.g., ALK/ROS1 rearrangements^{33,34} and EGFR mutations³⁵) in order to guide the implementation of effective targeted therapeutic strategies employing tyrosine kinase inhibitors (TKIs). Considering the profound risk associated with invasive tissue-based diagnostic approaches, clinicians increasingly prefer non-invasive diagnostic solutions³⁶ for both initial diagnosis and longitudinal monitoring of disease progression.³⁷ NanoVilli Chips were developed to harvest tumor-derived EVs to enable non-invasive characterization of tumors. The feasibility of quantifying the dynamic changes in both ROS1 rearrangements and the EGFR T790M mutations from tumor-derived EVs in NSCLC patients was assessed and these data were correlated with patient outcomes measured by radiographic imaging, which is the current gold standard for evaluating the therapeutic response of solid tumors clinically.

FIGS. 1A and 1B show an illustration showing the structures of intestinal microvilli and a schematic of a device and method according to an embodiment. Inspired by the distinctive structures of intestinal microvilli (FIG. 1A), which are densely packed on the intestinal walls to increase their intestinal mucosal surface areas for enhanced absorption, a biostructure-inspired NanoVilli Chip (FIG. 1B) featuring densely packed anti-epithelial cell adhesion molecule (anti-EpCAM)-grafted silicon (Si) nanowire arrays was designed and tested to achieve highly efficient and reproducible immunoaffinity capture of tumor-derived extracellular vesicles (EVs). A NanoVilli Chip is composed of (i) an anti-EpCAM-grafted Si nanowire substrate (SiNWS) and (ii) a superimposed polydimethylsiloxane (PDMS)-based chaotic mixer. Captured tumor-derived EVs are lysed in the device to release EV-derived RNA, which was extracted for downstream analysis via reverse transcription Droplet Digital™ PCR (RT-ddPCR). This workflow was utilized to detect gene alterations such as ROS1 rearrangements or epidermal growth factor receptor (EGFR) T790M mutations in non-small cell lung cancer (NSCLC) quantitatively.

Results and Discussion

Fabrication of NanoVilli Chips

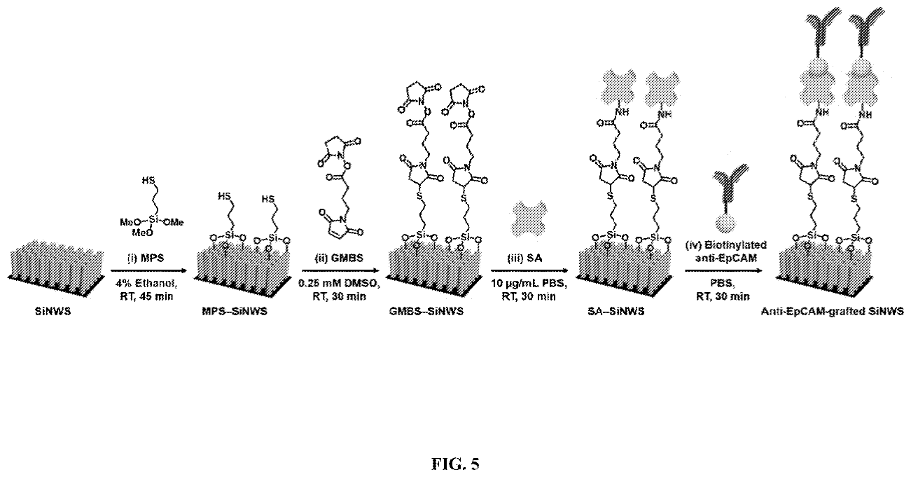

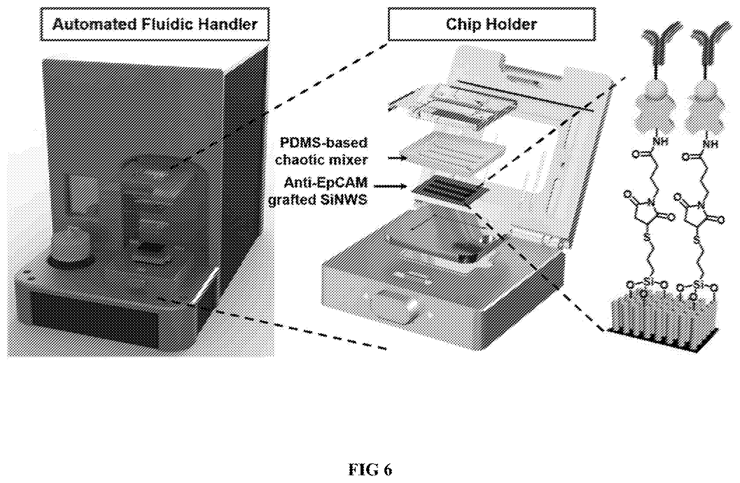

The nanostructures-embedded substrates (i.e., SiNWS) were fabricated via photolithography, followed by silver (Ag) nanoparticle-templated wet etching³⁸ to generate vertically aligned nanowire arrays on a Si wafer.³⁹ This fabrication process confers precise control over the diameters (100-200 nm), lengths (1-2 or 10-15 μm) and spacings (200-400 nm) of the Si nanowires (confirmed by scanning electron microscopy), resulting in large surface areas that enable enhanced immunoaffinity capture of tumor-derived EVs. A 4-step modification process was designed for the preparation of anti-EpCAM-grafted SiNWS (FIG. 5). Chaotic mixers were prepared by thermally curing PDMS pre-polymer (Sylgard 184) on a Si-based replicate mold (master wafer). On the mold, the herringbone patterns were fabricated by inductively coupled plasma-reactive ion etching (ICP-RIE). Compared to the SU-8 photolithographically deposited patterns used previously,⁴⁰ the ICP-RIE fabricated patterns on Si are much more durable over time with repeated usage. The chaotic mixing behavior in the devices were altered based on findings reported by Sheng et al.,⁴¹ where the spacings of herringbone patterns and the microchannel heights/widths/lengths (70 μm×2 mm×60 mm) were configured to optimize physical contact between anti-EpCAM-grafted SiNWS and tumor-derived EVs in plasma. Prior to EV capture studies, a custom-designed chip holder was employed to couple the PDMS-based chaotic mixers onto anti-EpCAM-grafted SiNWS to complete the chip assembly (FIG. 6). This chip holder also serves as an interface with syringe/syringe pumps used for handling plasma samples and reagents.

Characterization of Extracellular Vesicle Captured in NanoVilli Chips

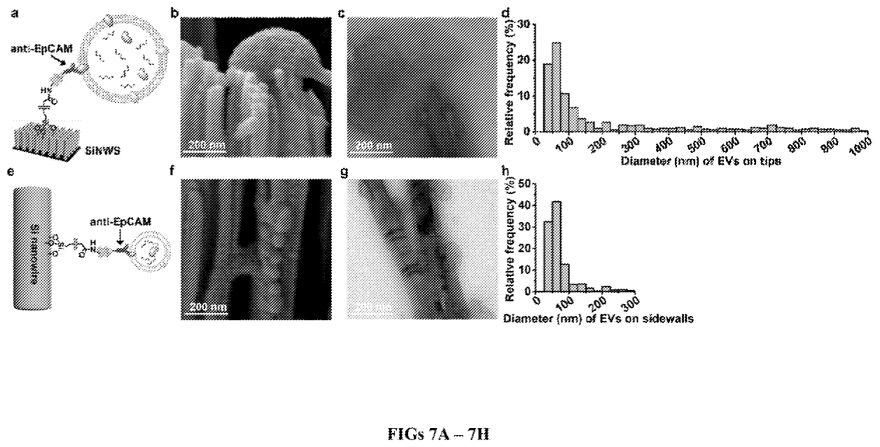

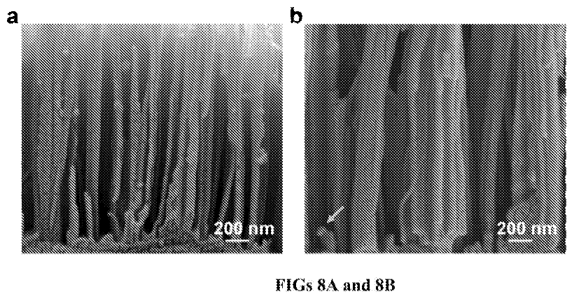

To study the function and performance of NanoVilli Chips, tumor-derived EVs were purified by ultracentrifugation from serum-free culture media of HCC78 NSCLC cells which harbor the SLC34A2-ROS1 rearrangement. These HCC78-derived EVs were first characterized by both dynamic light scattering (DLS) and TEM. The inset in FIG. 2A shows a typical TEM image of the HCC78-derived EVs after uranyl acetate negative staining. These EVs exhibited cup- or spherical-shaped morphologies with sizes ranging between 30 and 1000 nm. As a model system for testing NanoVilli Chips, artificial plasma samples were prepared by spiking aliquoted 10-μL HCC78-derived EVs into 90-μL freshly isolated healthy donor blood plasma. After EV capture, the NanoVilli Chips were disassembled to remove the PDMS-based chaotic mixers. To prepare samples for SEM imaging, the SiNWS underwent paraformaldehyde fixation, ethanol dehydration, and vacuum sputter coating with gold. The SiNWS were then cut to expose the cross sections of the Si nanowire arrays. The inset in FIG. 2B shows a cross-sectional SEM image of Si nanowires with HCC78-derived EVs (diameters=30-300 nm) adhered along the sidewalls of the nanowires. For samples characterized by TEM, Si nanowires with immobilized EVs were mechanically detached from the underlying substrate. The detached Si nanowires were collected and transferred onto TEM grids. The immobilized EVs along the sidewalls of Si nanowires range between 30 and 300 nm in diameter (FIG. 2C). Additionally, both SEM and TEM images showed that EVs with diameters greater than 300 nm were immobilized on the tips of SiNWS (FIGS. 7A-7D), which is expected, given that these EVs are too large to fit into the spacings (200-400 nm) between the Si nanowires. In comparison, negligible amounts of EVs were captured in control experiments where the anti-EpCAM capture agent was absent (FIGS. 8A and 8B). To confirm the identity of EVs, immunogold staining via anti-CD63 (an EV surface marker) was employed to label EVs with 10-nm gold nanoparticles before and after anti-EpCAM-based immunoaffinity capture onto Si nanowires, respectively. TEM images showed that both pre-capture and post-capture (FIGS. 2D and 2E) EVs could be decorated with 10-nm gold nanoparticles via anti-CD63.

The HCC78-derived EVs also express cytokeratin (CK) due to their epithelial origin, which enables immunohistochemical characterization of tumor-derived EVs immobilized on the SiNWS (FIGS. 2F-2H) via fluorescence microscopy (Nikon, 90i). As shown in FIGS. 2F and 2G, CK-positive EVs trapped on the tips of SiNWS were visualized by fluorescence microscopy. The actual size distribution of these EVs was determined by SEM (FIGS. 7B and 7D).

FIGS. 2A-2H are images, graphs, and schematics showing the characterization of tumor-derived extracellular vesicles (EVs) in solution and on anti-epithelial cell adhesion molecule (anti-EpCAM)-grafted silicon nanowire substrates (SiNWS) in NanoVilli Chips according to an embodiment. (FIG. 2A) Size distribution (n=653, diameters=30-1000 nm) of HCC78-derived EVs, measured by transmission electron microscopy (TEM). Inset: a representative TEM image (scale bar, 100 nm) of HCC78-derived EVs. (FIG. 2B) Size distribution of HCC78-derived EVs (n=425, diameters=30-300 nm) immunoaffinity-captured on the sidewalls of Si nanowires (SiNWs) measured by scanning electron microscopy (SEM). Inset: a representative cross-sectional SEM image (scale bar, 100 nm) of a device with immobilized HCC78-derived EVs. (FIG. 2C) A TEM image of HCC78-derived EVs immobilized on the sidewalls of Si nanowires. Scale bar, 100 nm. (FIG. 2D) Immunogold staining by anti-CD63 was employed to verify the identity of EVs captured on Si nanowires. (FIG. 2E) Schematic illustrating the immobilization of 10-nm gold nanoparticles via anti-CD63 on to a tumor-derived EV attached to the sidewall of a Si nanowire by anti-EpCAM. (FIG. 2F and FIG. 2G) Fluorescence microscopy images confirming the capture of HCC78-derived EVs immobilized on the SiNWS using an antibody to the epithelial tumor marker cytokeratin, CK. (FIG. 2H) Schematic depicting how anti-EpCAM and anti-CK were used for EV capture and immunostaining of CK, respectively.

Extracellular Vesicle-Derived RNA Assay Using NanoVilli Chips

To optimize EV-capture performance for NanoVilli Chips, different experimental parameters were examined, including flow rates, Si nanowire lengths, and anti-EpCAM concentrations. In each study, a 100-μL artificial plasma sample was run through a NanoVilli Chip. Subsequently, a TRIzol solution (Zymo Research, USA) was introduced into the device to lyse the captured EVs. The resulting lysate was subjected to RNA extraction using a Direct-zol™ RNA MicroPrep Kit (Zymo Research, USA), followed by treatment with DNase I to remove residual DNA. The extracted EV-derived RNA was then evaluated and quantified using a Qubit™ 3.0 Fluorometer in combination with the Qubit RNA HS Assay. The amount of the extract EV-derived RNA is denoted as RNA_{Cap-EV}. In parallel, 90-μL healthy-donor plasma samples were analyzed via the same workflow, where the systems' RNA background is denoted as RNA_{bg}. To determine the EV-capture efficiencies of NanoVilli Chips, RNA directly extracted from aliquoted 10-μL HCC78-derived EVs (that were not passed through a NanoVilli Chip) was directly quantified, labeled as RNA_{ori-EV}. The EV-capture performance of NanoVilli Chips was assessed by calculating the RNA recovery rate using the following equation:

$$RNA recovery rate = \frac{{RNA}_{cap \text{-} E V} - {RNA}_{b g}}{{RNA}_{ori \text{-} E V}} \qquad ( 1 )$$

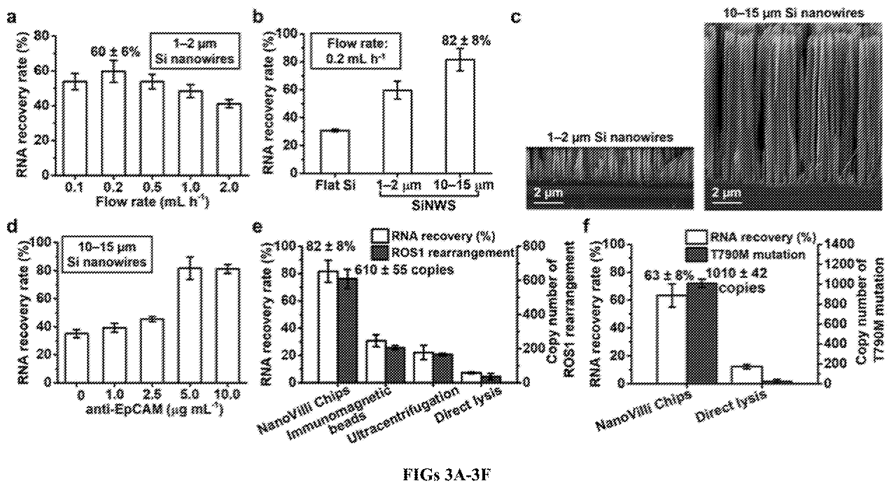

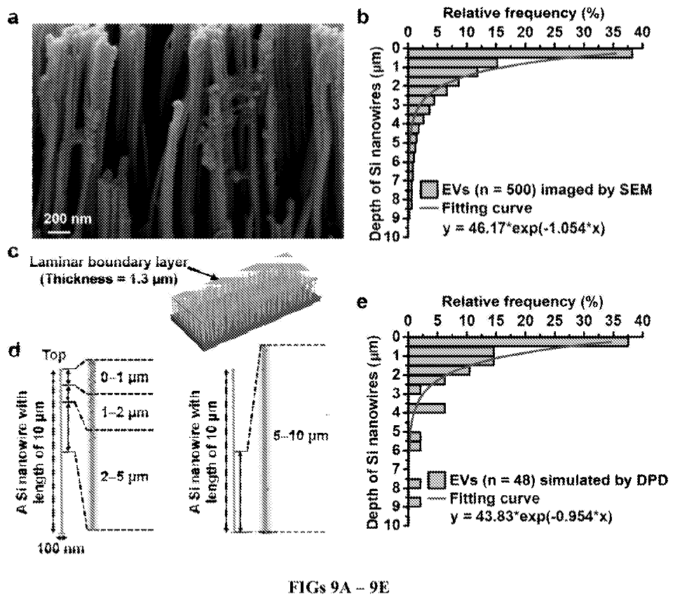

To study how flow rate affects EV-capture performance, 100-μL artificial plasma samples were injected into NanoVilli Chips (Si nanowire lengths=1-2 μm) at flow rates of 0.1, 0.2, 0.5, 1.0, and 2.0 mL h⁻¹. A flow rate of 0.2 mL h⁻¹ resulted in an optimal RNA recovery rate of 60±6% (FIG. 3A). This flow rate was subsequently used to investigate the relationship between Si nanowire length and EV-capture performance (FIG. 3B). NanoVilli Chips with long Si nanowires (lengths=10-15 μm) exhibited an 82±8% RNA recovery rate, which was significantly higher than the 60±6% and 31±1% observed for the devices with shorter Si nanowires (lengths=1-2 μm) and flat Si substrates, respectively. The lengths of the embedded Si nanowires NanoVilli Chips were verified by SEM (FIG. 3C). Additionally, SEM was also used to characterize how EVs (n=500, diameters=30-300 nm) distributed along the depth of Si nanowires (FIG. 9A). The results in FIG. 9B showed that 53.4%, 20.4%, 20.4%, 5.8%, and 0% of EVs were immobilized at depths of 0-1 μm, 1-2 μm, 2-5 μm, 5-9 μm, and 9-10 μm from the top of Si nanowires, respectively. This demonstrates that the increased surface area of the longer Si nanowires improves immunoaffinity capture of tumor-derived EVs such that a total length of 10 μm is sufficient.

To validate the results of the EV distribution observed by SEM, a computational simulation was conducted. The well-known laminar boundary layer effect⁴² dominates fluid behavior at the surface of microfluidic channels. These laminar boundary layers were estimated to be about 1.3 μm thick (FIG. 9C). Therefore, the flow velocity near the top of Si nanowire matrix is very slow and the EV diffusion into the Si nanowire matrix is primarily attributed to Brownian motion of EVs. A dissipative particle dynamics (DPD) simulation⁴³ was used to study the EV capture process by the Si nanowire matrix when the EVs diffuse from the top to the bottom of Si nanowire matrix (FIG. 9D). As shown in FIG. 9E, 52.1%, 25.0%, 14.6%, 8.3% and 0% of EVs were located at depths of 0-1 μm, 1-2 μm, 2-5 μm and 5-9 μm, 9-10 μm from the top of Si nanowires, respectively. An empirical function with the exponential form was used to describe the EV distribution probability profiles along the depth of Si nanowire. The results derived from the experiment and DPD simulation were very close.

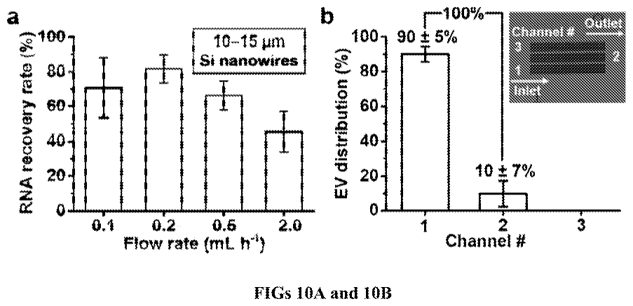

It was next attempted to reduce the consumption of the EV-capture agent (anti-EpCAM) without compromising the EV-capture performance at the optimal flow rate and nanowire configurations identified earlier. Five different biotinylated anti-EpCAM concentrations (i.e., 0.5, 1.0, 2.5, 5.0 and 10.0 μg mL⁻¹) were tested for SiNWS modification. At concentrations <5.0 μg mL⁻¹ EV-capture performance was reduced dramatically (FIG. 3D). When the concentration was higher than 5.0 μg mL⁻¹, the EV-capture performance did not improve, indicating that 5.0 μg mL⁻¹ is sufficient to capture EVs in NanoVilli Chips. Extracellular vesicle capture capacity along the channel was tested by segmentally quantifying RNA recovery rates of the three channels (FIG. 10B). The results indicated that 90% of the EVs were captured in the first channel of the NanoVilli Chips.

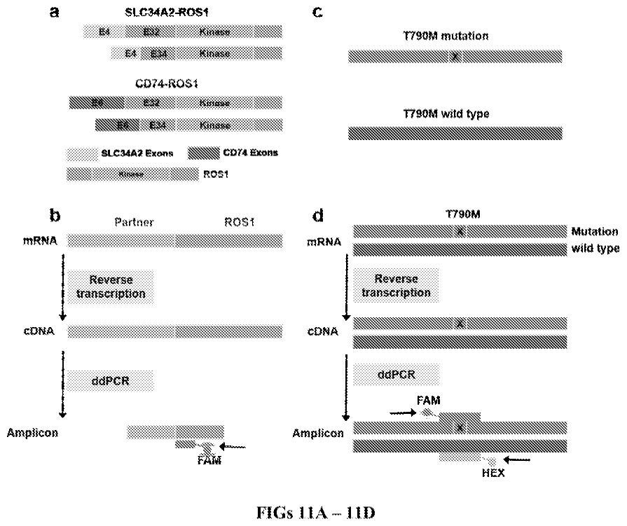

With the optimal EV-capture conditions identified, the performances of NanoVilli Chips with two commonly used EV enrichment methods was compared (i.e., immunomagnetic beads¹⁸ and ultracentrifugation¹⁷) using the artificial plasma samples. Since HCC78 NSCLC cells harbor specific SLC34A2-ROS1 rearrangement, these artificial plasma samples can be used to validate the feasibility of detecting ROS1 rearrangement in the EVs captured by NanoVilli Chips. In parallel with the RNA quantification, matching RNA samples obtained from the three EV enrichment methods were subjected to the RT-ddPCR assay to quantify the ROS1 rearrangement copy number (FIGS. 11A-11D). Results summarized in FIG. 3E indicate that NanoVilli Chips exhibited a superior RNA recovery rate of 82±8% compared to the 31±4% and 22±5% observed for immunomagnetic beads and ultracentrifugation, respectively. Consistent performance in detecting ROS1 rearrangements (610±55, 206±12, and 165±8 copies) when comparing NanoVilli Chips, immunomagnetic beads, and ultracentrifugation was observed. For control purposes, the artificial plasma samples were directly processed and subjected to the RT-ddPCR assays. The resultant low RNA recovery rate (7±1%) may due to the RNase and proteins in the background plasma had a negative effect on the RNA quality during direct lysing process, highlighting the necessity of EV enrichment for reliable EV-based RNA analysis. Finally, the general applicability of NanoVilli Chips for enriching NSCLC-derived EVs using different artificial plasma samples containing EVs purified from NCI-H1975 cells (harboring EGFR T790M point mutation) was evaluated. As shown in FIG. 3F, an EV-capture efficiency of 63±8% was measured with the NanoVilli Chip, which is significantly higher than the 12±2% observed following a direct lysis method. Using RT-ddPCR, 1010±42 copies of EGFR T790M mutation were detected in enriched EV-derived RNA (whereas 27±17 copies of EGFR T790M mutation were observed for the direct lysis method). Overall, the optimized conditions developed for NanoVilli Chips enabled efficient purification of tumor-derived EVs from artificial plasma samples with capture efficiencies ranging from 63 to 82% in a period of 30 min.

FIGS. 3A-3F are graphs and images showing optimization of NanoVilli Chips for immunoaffinity capture of tumor-derived extracellular vesicles (EVs) using artificial plasma samples according to an embodiment. (FIG. 3A) The EV-capture performance of NanoVilli Chips (Si nanowires lengths=1-2 μm) was studied at flow rates of 0.1, 0.2, 0.5, 1.0, and 2.0 mL h⁻¹. (FIG. 3B) Extracellular vesicle-capture performance observed for three different control groups: flat Si substrates, short Si nanowires (lengths=1-2 μm), and long Si nanowires (lengths=10-15 μm). (FIG. 3C) Scanning electron microscope (SEM) images (scale bar, 2 μm) of the two NanoVilli Chips with different lengths of embedded Si nanowires (1-2 vs. 10-15 μm). (FIG. 3D) The EV-capture performance observed for NanoVilli Chips with Si nanowires (lengths=10-15 μm) coated by biotinylated anti-epithelial cell adhesion molecule (anti-EpCAM) at concentrations of 0, 1.0, 2.5, 5.0, and 10.0 μg mL⁻¹. (FIG. 3E) The RNA recovery rate and copy numbers of ROS1 rearrangements observed for NanoVilli Chips, immunomagnetic beads and ultracentrifugation. As a control, the artificial plasma samples were directly subjected to RT-ddPCR analysis to the necessity of conducting EV enrichment. (FIG. 3F) General applicability of NanoVilli Chips was validated using different artificial plasma samples containing EVs purified from NCI-H1975 NSCLC cells harboring epidermal growth factor receptor (EGFR) T790M mutation.

Non-Invasive Detection of Gene Alterations in Non-Small Cell Lung Cancer Patients

The NanoVilli Chips were operated at the optimal conditions identified in the initial studies to enrich tumor-derived EVs from NSCLC patient blood plasma samples. A cohort of 13 NSCLC patients—seven harboring a clinically confirmed CD74-ROS1 rearrangement (treatment naïve, stages III-IV) and six with an acquired EGFR T790M mutation (resistant to the prior EGFR-TKI treatment, i.e., gefitinib or erlotinib, stages III-IV) were recruited for this feasibility study (Table 1). Control studies were performed in parallel on nine healthy donors. In each study, 200 μL, samples of processed plasma were run through a NanoVilli Chip. For the 7 ROS1-rearranged NSCLC patients, 18 to 468 copies of the CD74-ROS1 rearrangement were detected in the NanoVilli Chip-enriched EVs at diagnosis. For the six EGFR T790M-mutated NSCLC patients, 0 to 225 copies of the acquired EGFR T790M mutation were detected at their time of disease relapse. In the control studies, all of the nine healthy donors were negative for both ROS1 rearrangement and EGFR T790M mutation (Table 1).

TABLE 1
Clinical characteristics of non-small cell lung cancer (NSCLC,
adenocarcinoma) patients and healthy donors (HD) enrolled in this study.

| | | | | | | | Copy numbers of T790M |
|---|---|---|---|---|---|---|---|
| | | Age | Smoking | Tumor | Clinical | Gene status | mutation/ROS1 |
| Patient No. | Gender | (Y) | history (Y) | grade | stage | (Tissue)^{b} | rearrangement in EVs^{a} |
| R01 | Female | 62 | None | 3 | IIIB | CD74-ROS1 | 18 |
| R02 | Male | 41 | None | 2 | IIIB | CD74-ROS1 | 27 |
| R03 | Male | 61 | 35 | 2 | IV | CD74-ROS1 | 54 |
| R04 | Male | 34 | None | 3 | IV | CD74-ROS1 | 54 |
| R05 | Male | 61 | 35 | 2 | IV | CD74-ROS1 | 99 |
| R06 | Male | 34 | None | 3 | IV | CD74-ROS1 | 396 |
| R07-1 | Male | 32 | None | 3 | IV | CD74-ROS1 | 324 |
| R07-2 | Male | 32 | None | 3 | IV | N/A | 0 |
| R07-3 | Male | 32 | None | 3 | IV | N/A | 468 |
| E01 | Female | 55 | None | 3 | IV | T790M | 36 |
| E02 | Female | 62 | None | 3 | IV | T790M | 36 |
| E03 | Male | 66 | None | 3 | IV | T790M | 90 |
| E04-1 | Male | 53 | None | 3 | IV | N/A | 0 |
| E04-2 | Male | 53 | None | 3 | IV | T790M | 225 |
| E04-3 | Male | 53 | None | 3 | IV | N/A | 9 |
| E05 | Male | 62 | None | 2 | IV | T790M | 72 |
| E06 | Male | 61 | None | 2 | IV | T790M | 81 |
| HD01 | Male | 30 | None | N/A | N/A | N/A | 0 |
| HD02 | Male | 26 | None | N/A | N/A | N/A | 0 |
| HD03 | Male | 29 | None | N/A | N/A | N/A | 0 |
| HD04 | Male | 46 | None | N/A | N/A | N/A | 0 |
| HD05 | Female | 36 | None | N/A | N/A | N/A | 0 |
| HD06 | Male | 32 | None | N/A | N/A | N/A | 0 |
| HD07 | Male | 56 | None | N/A | N/A | N/A | 0 |
| HD08 | Female | 58 | None | N/A | N/A | N/A | 0 |
| HD09 | Male | 60 | None | N/A | N/A | N/A | 0 |

^{a}per 0.2 mL plasma.
^{b}N/A: not available.

Dynamic Monitoring of Gene Alterations Over the Course of Treatment Intervention

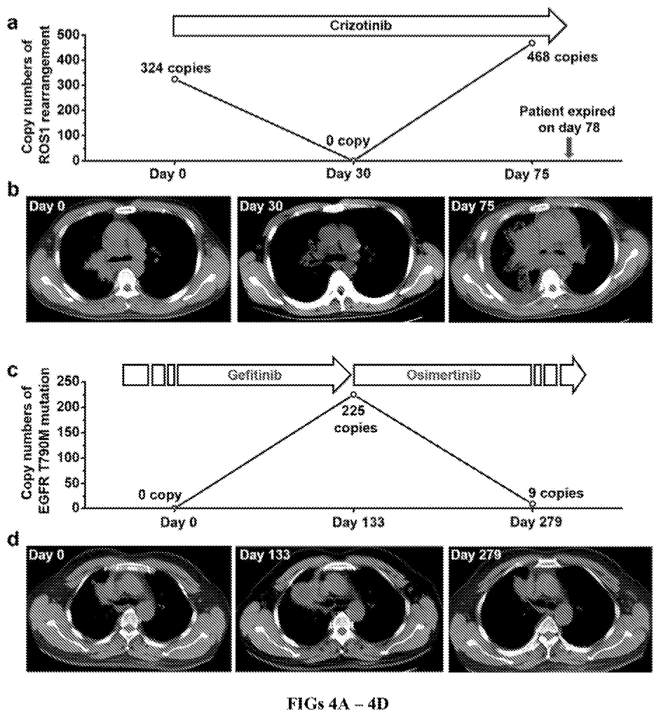

The feasibility of combining tumor-derived EV enrichment by NanoVilli Chips and RT-ddPCR to monitor dynamic changes in disease course during treatment was evaluated. Serial blood draws were obtained from patient R07 with the ROS1 rearrangement before and after crizotinib treatment. The copy numbers of rearranged ROS1 are plotted in FIG. 4A. Matching serial computed tomography (CT) images depict the lesions in the patient's chest on days 0, 30, and 75 post-crizotinib treatment. The patient was found to have a partial response on day 30 but unfortunately relapsed after day 75 (FIG. 4B). The patient died of untreatable tumor burden on day 78. Dynamic changes in ROS1 copy numbers were consistent with CT imaging and clinical outcomes, suggesting that this EV-based mRNA assay may serve as a complementary diagnostic tool for monitoring treatment outcomes in NSCLC patients with ROS1 rearrangements. In FIG. 4C, patient E04 was tracked over a period of 279 days. Blood was collected serially at three time points: day 0, when the patient had responded to a first-generation EGFR-TKI (i.e., gefitinib); day 133, when the patient acquired resistance; and day 279, when the patient had a partial response after 146 days of treatment with the third-generation EGFR-TKI osimertinib, which targets the acquired EGFR T790M mutation. The emergence of the acquired EGFR T790M mutation (copy number increased from 0 to 225) between days 0 and 133 indicated resistance to the initial therapy, suggesting a possible timepoint for switching from gefitinib to osimertinib. At 146 days post-osimertinib treatment, the EGFR T790M copy numbers decreased from 225 to 9, consistent with the tumor shrinkage observed via CT imaging (FIG. 4D). The US Food and Drug Administration has approved osimertinib for the treatment of patients with an acquired EGFR T790M mutation who progress during prior EGFR-TKIs treatments.⁴⁴ Confirmation of the EGFR T790M mutation by tissue re-biopsy is required for treatment selection after relapse from prior EGFR-TKIs treatments. These results suggest the NanoVilli Chip-based tumor-derived EV capturing platform is compatible with detecting both gene rearrangements (i.e., ROS1 rearrangements) and gene mutations (i.e., EGFR T790M mutation) for monitoring early treatment response and guiding the selection of alternative therapies non-invasively.

FIGS. 4A-4D are images and graphs demonstrating that NanoVilli Chips combined with reverse transcription Droplet Digital™ PCR (RT-ddPCR) analysis can be used to detect and to monitor ROS1 rearrangements or acquired EGFR T790M mutation in tumor-derived EVs purified from non-small cell lung cancer (NSCLC) patients' blood according to an embodiment. (FIG. 4A) The dynamic change (0 to 75 days) of the ROS1 rearrangements observed for patient R07 with CD74-ROS1 rearrangement before and after crizotinib treatment. (FIG. 4B) Chest computed tomography (CT) scans taken at days 0, 30, 75 post-crizotinib treatment. (FIG. 4C) The dynamic changes (0 to 279 days) of EGFR T790M mutation observed for patient E04 before and after osimertinib treatment. (FIG. 4D) Chest CT images were taken at day 0 (following response to gefitinib treatment), day 133 (disease relapse), and day 279 (post-treatment with osimertinib).

Conclusions and Prospects

A bio-inspired device capable of highly efficient and reproducible immunoaffinity capture of tumor-derived EVs from blood plasma samples has been successfully developed and demonstrated. The anti-EpCAM-grafted Si nanowire arrays that comprise these NanoVilli Chips mimic the distinctive structures of intestinal microvilli, providing dramatically increased surface area for capturing tumor-derived EVs. A PDMS-based microfluidic chaotic mixer is used to establish direct physical contact between tumor-derived EVs and anti-EpCAM-grafted SiNWS, further enhancing the EV-capture performance. The influence of flow rate, length of Si nanowires, and anti-EpCAM concentrations to identify conditions that yield optimal EV-capture performance were evaluated. When operated at these optimal conditions, NanoVilli Chips enable highly efficient, reproducible and rapid (30 min) enrichment of tumor-derived EVs from both artificial plasma samples as well as plasma samples isolated from NSCLC patients. By coupling NanoVilli Chips with a downstream RT-ddPCR, a new type of EV-based mRNA assay for quantitatively detecting and monitoring targetable oncogenic gene alterations has been developed. In clinically relevant applications, tumor-derived EVs captured on NanoVilli Chips can provide critical diagnostic information as a source for detecting specific oncogenic gene alterations that correlate with treatment responses and disease progression to inform the clinical management of NSCLC patients.

Experimental Section

Fabrication of Anti-EpCAM-Grafted Silicon Nanowire Substrate. First, thiol groups were introduced onto SiNWS by exposure to (3-mercaptopropyl) trimethoxysilane (MPS, 211.4 mg, 200 μL, Sigma-Aldrich, USA) vapor at room temperature for 45 min. The SiNWS were rinsed with ethanol three times to wash off unbound reagents. Second, freshly prepared MPS-SiNWS were incubated with the N-maleimidobutyryl-oxysuccinimide ester (GMBS, 0.25 mM in DMSO, Sigma-Aldrich, USA) solution for 30 min to attach GMBS on the surface of SiNWS. Third, GMBS-SiNWS were reacted with streptavidin (SA, 10 μg mL⁻¹, Thermo Fisher Scientific, USA) solution at room temperature for 30 min to immobilize SA. The obtained SA-SiNWS were rinsed with 1× phosphate-buffered saline (PBS, pH 7.4, Thermo Fisher Scientific) to remove excess SA. Fourth, to graft anti-EpCAM onto the SA-SiNWS, biotinylated anti-EpCAM (Abcam, USA) at concentrations of 1.0, 2.5, or 5.0 μg mL⁻¹ in PBS (100 μL) was incubated on the SA-SiNWS for 30 min at room temperature. After rinsing off the unbounded biotinylated anti-EpCAM, the anti-EpCAM-grafted SiNWS were blocked with 5% bovine serum albumin (BSA, Thermo Fisher Scientific) solution for 30 min. The total inner volume of 3 microfluidic channels in a NanoVilli Chip was 20 μL.

Culture of Non-Small Cell Lung Cancer Cell Lines. Non-small cell lung cancer (NSCLC) cell lines including HCC78 and NCI-H1975 were obtained from the American Type Culture Collection and regularly tested and found negative for mycoplasma contamination. These NSCLC cells were cultured in RPMI-1640 growth medium (Thermo Fisher Scientific, USA) with 10% (v/v) fetal bovine serum (FBS, Thermo Fisher Scientific), 1% (v/v) GlutaMAX-I (Thermo Fisher Scientific), and penicillin-streptomycin (100 U mL⁻¹, Thermo Fisher Scientific) in a humidified incubator with 5% CO₂ at 37° C.

Preparation and Isolation of Non-Small Cell Lung Cancer Cell-Derived Extracellular Vesicles. Both HCC78 and H1975 NSCLC cells were grown in 18 Nunc™ EasYDish™ dishes (145 cm², Thermo Fisher Scientific) for three days. The cells were then cultured in serum-free medium (Thermo Fisher Scientific) for 24-48 h. Thereafter, the culture medium was collected for centrifugation at 300 g (4° C.) for 10 min to remove cells and cell debris. The supernatants were transferred to new Falcon™ 50 mL Conical Centrifuge Tubes (Thermo Fisher Scientific) and centrifuged at 2800 g (4° C.) for 10 min to eliminate remaining cellular debris and large particles. The supernatants were carefully transferred into Ultra-Clear Tubes (38.5 mL, Beckman Coulter, Inc., USA), followed by ultracentrifugation using Optima™ L-100 XP Ultracentrifuge (Beckman Coulter, Inc, USA) at 100,000 g (4° C.) for 70 min. After removing the supernatant, EV pellets at the bottom of the tubes were resuspended into 400 μL of PBS (Thermo Fisher Scientific) and were stored at −80° C. for future use.

Preparation of Artificial Plasma Samples Containing Non-Small Cell Lung Cancer Cell-Derived Extracellular Vesicles. The plasma was isolated from the blood samples of healthy donors with approval from the UCLA Institutional Review Board (IRB, #00000173). Artificial plasma samples (each had a total volume of 100 μL) were prepared by spiking 10 of NSCLC cell-derived EVs (see above) into 90 μL of healthy-donor plasma.

Capture of Tumor-Derived Extracellular Vesicles on NanoVilli Chips. Prior to the injection of artificial plasma samples, 200 μL of PBS was introduced into a NanoVilli Chip via an automated digital fluidic handler at a flow rate of 0.5 mL h⁻¹ to test for leaks. Next, 100 μL of artificial plasma or blood plasma containing tumor-derived EVs was introduced into the NanoVilli Chip at an optimal flow rate of 0.2 mL h⁻¹. For the optimization of flow rates, replicates of 100 μL of artificial plasma samples were introduced into NanoVilli Chips at flow rates of 0.2, 0.5, 1.0, and 2.0 mL h⁻¹, respectively.

Characterization of the Embedded Silicon Nanowires and Captured Extracellular Vesicles by Scanning Electron Microscopy. To characterize the Si nanowires embedded in the SiNWS, the SiNWS were cut to expose the cross sections of the silicon nanowire arrays. The broken SiNWS was placed on the SEM sample holder for SEM imaging (ZEISS Supra 40VP SEM at an accelerating voltage of 10 keV). For SEM characterization of EVs captured on Si nanowires, the SiNWS were separated from the NanoVilli Chip after capturing EVs from 100 μL of artificial plasma samples. The EVs immobilized on SiNWS were fixed in 4% paraformaldehyde for 1 h. The samples were dehydrated by sequential immersion in 30, 50, 75, 85, 95, and 100% ethanol solutions for 10 min per solution. After overnight lyophilization, sputter-coating with gold was performed at room temperature. The morphology of EVs immobilized on Si nanowires were observed using a ZEISS Supra 40VP SEM at an accelerating voltage of 10 keV.

Transmission Electron Microscopy Characterization of HCC78-Derived Extracellular Vesicles. The HCC78-derived EVs in solution or captured by the Si nanowires were fixed in 4% paraformaldehyde (PFA) for 30 min prior to morphological characterization and determining the size distribution of tumor-derived EVs via TEM. Afterward, the EV samples were deposited onto 200-mesh formvar and carbon coated copper grids and incubated for 5 min. After wiping off the excess sample, the grids were treated with 2% uranyl acetate for 10 min and then washed 3 times with deionized water. Grids were dried for TEM imaging by JEM1200-EX (JEOL USA Inc.) at 80 kV. To verify the identity of EVs in solution and captured on Si nanowires, immunogold staining by anti-CD63 was employed for TEM imaging. Fixed EVs in solution or captured on Si nanowires were applied to 200-mesh formvar and carbon-coated nickel grids and incubated for 5 min before being wiped off from the grids. Then, grids were incubated in a blocking solution (0.4% BSA in PBS) for 30 min and then rinsed 3 times using deionized water. Thereafter, grids were incubated with mouse anti-CD63 (Abcam, USA, positive control) or with blocking solution (negative control) for 1 h. After being rinsed 3 times with deionized water, the grids were incubated with goat anti-mouse IgG H&L 10-nm gold (Abcam, USA) for 1 h. After again being rinsed 3 times using deionized water, the grids were negatively stained using 2% uranyl acetate and then dried for TEM imaging using a JEM1200-EX (JEOL USA Inc.) at 80 kV.

Immunostaining by anti-CK and Fluorescence Characterization of Tumor-Derived EVs Immobilized on Silicon Nanowire Substrates. Tumor-derived EVs immobilized on SiNWS were fixed with 4% PFA for 10 min, followed by incubation with 0.1% Triton X 100 in PBS for 10 min at room temperature. Then they were incubated with a PBS solution containing Pan-CK antibody (Abcam, USA, 1:100 (v/v)) and Normal Donkey serum (Jackson ImmunoResearch, USA, 2%) at 4° C. overnight. After being washed with PBS 3 times, the tumor-derived EVs captured on SiNWS were further incubated with Donkey anti-Rabbit IgG (H+L) (Alexa Fluor 488, Thermo Fisher Scientific, USA, 1:500 (v/v)) for 1 h. After washing off the excess reagent, the tumor-derived EVs immobilized on SiNWS were characterized using a fluorescence microscope (Nikon 90i, exposure time=200 ms).

Extraction of RNA from Tumor-Derived EVs Captured on NanoVilli Chips. To extract RNA from tumor-derived EVs captured on NanoVilli Chips, on-chip lysis of EVs was performed by introducing 600 μL of TRIzol solution (Zymo Research, USA) and 600 μL of anhydrous ethanol (Sigma-Aldrich) sequentially through the NanoVilli Chip. The effluent solution was collected in a 2.0 mL RNase-free Eppendorf tube at the same time. Then, RNA was purified using a Direct-zol™ RNA MicroPrep Kit (Zymo Research). The enzyme DNase I was used to digest DNA for 15 min to make sure that cfDNA was not analyzed in the measurements. The RNA was dissolved in DNase/RNase-free water and then measured with a Qubit™ 3.0 Fluorometer (Thermo Fisher Scientific) in combination with the Qubit RNA HS Assay (Thermo Fisher Scientific) using the manufacturer's protocol.

Quantification of ROS1 Rearrangements or EGFR T790M Mutation from Extracellular Vesicle-Derived mRNA by RT-ddPCR. Extracellular vesicle-derived mRNA was reverse-transcribed to cDNA using a Maxima H Minus Reverse Transcriptase Kit (Thermo Fisher Scientific). The EV-derived mRNA was added into a reaction solution containing 1×RT Buffer, dNTPs (0.5 mM), Random Hexamer (8 Maxima H Minus Reverse Transcriptase (6.5 U μL⁻¹) and RNase inhibitor (1 U μL⁻¹). The reaction was run at 55° C. for 30 min and then 85° C. for 5 min. The cDNA generated from EV-derived mRNA was detected by the PrimePCR™ ddPCR™ Expert Design Assay Kit (dHsaEXD73338942, ROS1 rearrangements) or PrimePCR™ ddPCR™ Mutation Assay Kit (dHsaCP2000020, EGFR T790M mutation, Bio-Rad, USA) according to the manufacturer's instructions. For ddPCR, droplets were generated within a DG8™ Cartridge which was pre-loaded with sample (20 μL) and droplet generation oil (70 μL) for each sample. All droplets were transferred into a 96-well plate accordingly and sealed with a PX1 PCR Plate Sealer. A programmed Thermal Cycler was set at 96° C. for 10 min, followed by 40 cycles of 94° C. for 30 s and 60° C. for 60 s, and finally 98° C. for 10 min. The droplets containing amplicons were quantified with a QX200 Droplet Reader using the QuantaSoft™ software package.

Collection of Blood Plasma Samples from Non-Small Cell Lung Cancer Patients and Healthy Donors. Blood samples were collected from 12 NSCLC patients in Guangdong Provincial Hospital of Traditional Chinese Medicine and 9 healthy donors at UCLA in accordance with the Institutional Review Board (IRB). 6 NSCLC (stages III and IV) patients with known ROS1 rearrangements⁴⁵ were enrolled from October 2016 to June 2017 and 6 NSCLC patients with known EGFR T790M mutation from January 2018 to June 2018. Blood samples were centrifuged at 300 g for 5 min and then 2000 g for 5 min at 4° C. Plasma was collected and stored at −80° C. For each blood plasma sample, 200 μL of plasma was directly run through a NanoVilli Chip.

Fabrication of Silicon Nanowire Substrates

Silicon nanowire substrates (SiNWS) were fabricated via photolithography followed by silver (Ag) nanoparticle-templated wet etching^{S1} to introduce vertically aligned silicon (Si) nanowires onto Si wafers.^{S2-S5} First, a thin film photoresist (AZ 5214, AZ Electronic Materials USA Corp., Branchburg, N.Y., USA) was spin-coated onto a p-type Si(100) wafer (Silicon Quest, San Jose, Calif., USA) with resistivity of ca. 10-20 Ω·cm. After being exposed to ultraviolet (UV) light, the Si wafer was immersed into the etching solution containing hydrofluoric acid (HF, 4.6 M, Sigma-Aldrich, USA), silver nitrate (AgNO₃, 0.2 M, Sigma-Aldrich, USA) and deionized water. The lengths of Si nanowires were controlled by the etching duration.^{S1} Then, the Si wafer was immersed in boiling aqua regia (hydrochloric acid (HCl)/nitric acid (HNO₃), 3:1 (v/v), Sigma-Aldrich) for 15 min to remove the silver film. The obtained SiNWS were rinsed with acetone (>99.5%, Sigma-Aldrich) and then anhydrous ethanol (<0.005% water, Sigma-Aldrich) several times to remove the patterned photoresist. After being rinsed by deionized water and then dried by nitrogen, the nanowire structures on the surface of the Si substrate were ready for subsequent modification.

Chemical Modification to Prepare Anti-EpCAM-Grafted Silicon Nanowire Substrates

FIG. 5 is a scheme illustrating the surface chemical modification process used to prepare anti-epithelial cell adhesion molecule (anti-EpCAM)-grafted silicon nanowire substrates (SiNWS) according to an embodiment. (i) Surface silanization of SiNWS with (3-mercaptopropyl)trimethoxysilane (MPS) to form MPS-SiNWS. (ii) Incorporation of N-maleimidobutyryl-oxysuccinimide ester (GMBS) onto the surface of MPS-SiNWS. (iii) Immobilization of streptavidin (SA) onto GMBS-SiNWS. (iv) Conjugation of biotinylated anti-EpCAM onto SA-SiNWS.

Fabrication of Polydimethylsiloxane Chaotic Mixers

Polydimethylsiloxane (PDMS) chaotic mixers with herringbone features were molded from a master wafer prepared by inductively coupled plasma-reactive ion etching (ICP-RIE).^{S3,S6} A 100-μm-thick layer of negative photoresist (MicroChem Corp., USA) was spin coated onto a 3-inch silicon wafer and then exposed to UV light using a photomask with a serpentine rectangular microfluidic channel (20 mm length and 2.4 mm width). A second 35-μm-thick layer of negative photoresist was spin coated onto the wafer. Between the yet to be imprinted and previously exposed pattern, a second photomask with herringbone ridge features was aligned via a Mask Aligner (Karl Suss America Inc., USA). After the Si master was exposed to trimethylchlorosilane vapor for 1 min, the master was transferred to a Petri dish. The Petri dish was filled with the well-mixed PDMS prepolymer (RTV 615 A and B in 10 to 1 ratio, GE Silicones, USA), de-gassed, and then incubated in an oven at 80° C. for 48 h. This formed the 5 mm-thick PDMS microfluidic chaotic mixer, which was then peeled from the silicon master wafer/mold. Two through-holes were punched at the ends of the channel for insertion of tubing.

Photograph and Schematic of the NanoVilli Device

FIG. 6 is a photograph and a schematic showing the setup of the entire NanoVilli device according to an embodiment.

Characterization of Pre-Capture Extracellular Vesicles by Dynamic Light Scattering

Dynamic light scattering (DLS) was used to characterize the size distribution of HCC78-derived extracellular vesicles (EVs) in solution. For these studies HCC78-derived EVs were placed into a disposable microcuvette and analyzed using a Zetasizer Nano instrument (Malvern Instruments Ltd., UK) at room temperature.

Electron Microscopy Characterization of Post-Capture Extracellular Vesicles

FIGS. 7A-7H are images and graphs showing tumor-derived extracellular vesicles (EVs) captured on anti-epithelial cell adhesion molecule (anti-EpCAM)-grafted silicon nanowire substrates (SiNWS) characterized using scanning electron microscopy (SEM) and transmission electron microscopy (TEM) according to an embodiment. Scale bars, 200 nm. (FIG. 7A) Schematic illustration of an EV immobilized on tips of Si nanowires. (FIG. 7B and FIG. 7C) SEM and TEM images of EVs (sizes >300 nm) captured on the tips of Si nanowires. (FIG. 7D) Diameters (nm) of EVs (n=415) captured on the tips of Si nanowires measured by SEM. (FIG. 7E) Schematic illustration of an EV immobilized on the sidewall of a Si nanowire. (FIGS. 7F and 7G) SEM and TEM images of EVs (sizes <300 nm) immobilized on the sidewalls of Si nanowires. (FIG. 7H) Diameter (nm) of EVs (n=425) captured on the sidewalls of Si nanowires measured by SEM.

Comparison of Extracellular Vesicle Capture Performance on a NanoVilli Chip and a Control Device without Antibody Conjugation

FIGS. 8A and 8B are scanning electron microscopy (SEM) images showing the different extracellular vesicle (EV) capture performance on (FIG. 8A) a standard NanoVilli Chip (which is conjugated with anti-EpCam) and (FIG. 8B) a control device (a NanoVilli Chip without antibody conjugation) according to an embodiment. Scale bars, 200 nm. Very few EVs can be captured by the control device without anti-EpCAM-mediated immunoaffinity capture, as compared to many EVs captured on the NanoVilli Chip.

Extracellular Vesicle Distribution Probability Profiles Along the Depth of Si Nanowires Analyzed by Scanning Electron Microscopy and Computational Simulation

Scanning Electron Microscopy Characterization of Extracellular Vesicle distribution. To determine how EVs distribute along the depth of Si nanowires, SEM was employed to analyze EVs (n=500). FIG. 9A showed a representative SEM image of EVs (diameter=30-300 nm) captured by the Si nanowires. By calculating the relative frequencies of EVs located at different depths from the top of Si nanowires, a frequency distribution histogram (FIG. 9B) was obtained with results showing that 53.4%, 20.4%, 20.4%, 5.8%, and 0% of EVs were immobilized at the depths of 0-1 μm, 1-2 μm, 2-5 μm, 5-9 μm, and 9-10 μm from the top of Si nanowires, respectively. An empirical function with the exponential form shown in Eq. (S1) was used to describe the EV distribution probability profiles along the depth of Si nanowire from the experiment.

$$Probablity ( x ) = {x}_{0} \times exp ( \frac{x}{τ} ) \qquad ( S 1 )$$

where x is the depth from the top of the Si nanowire, x₀ is the pre-exponential factor, and τ is a constant with the unit of μm that indicates the mean depth, at which the EV distribution probability is reduced to 0.368 (about 1/e) times the value of x₀.

Using the obtained curve fitting formula y=46.17×exp(−1.054 x), the τ value was calculated as 0.949 μm, while the x₀ values are 46.17 for experiment.

Laminar Boundary Layers Thickness Calculation. The well-known laminar boundary layer effect⁷ dominates fluid behavior at the surface of any microfluidic channel. The laminar boundary layer thickness was calculated using Von Kaman laminar boundary layer thickness (δ₁) as shown in Eq. (S2):

$${δ}_{1} = {\int }_{0}^{∞} ( 1 - \frac{u ( y )}{{u}_{0}} ) dy \qquad ( S2 )$$

where u₀ is the mean velocity, and u(y) is the velocity component along the channel height, which can be calculated using Eq. (S3):

$$u ( y ) = \frac{1}{2 μ} \frac{dp}{dx} ( {y}^{2} - h y ) \qquad ( S3 )$$

where μ is the flow viscosity,

$$\frac{d p}{d x}$$

is the pressure drop, and h is the height of the channel.

The boundary layer thickness parameters

$$( {u}_{0} = 0.3 \; 97 mm {s}^{- 1} , μ = 0.0036 kg m - {s}^{- 1} , \frac{d p}{d x} = 35 Pa , h = 70 μm )$$

were used in the Von Kaman equation, and the laminar boundary layers were estimated to be about 1.3 μm thick. Therefore, the flow velocity near the top of Si nanowire matrix is slow (in a “no-slip condition”) and the EV diffusion into the Si nanowire matrix is attributed to Brownian motion of EVs.

Dissipative Particle Dynamics Simulation Method and Results. To include the Brownian mechanism in the system, the dissipative particle dynamics (DPD) simulation^{S8} was used to study the EV capture process by the Si nanowire matrix when the EVs diffuse from the top to the bottom of Si nanowire matrix. Unlike most other molecular simulation theories, only the repulsive force between beads was considered in a DPD system. Consequently, a DPD simulation can predict the equilibrated structure quickly and can keep some important atomistic information. In the DPD simulation, the movements of beads follow Newton's equation of motion. Considering the interaction between bead i and all its nearest beads j, the net force fᵢ imposed on bead i includes Fᵢⱼ^{c}, the conservative force, Fᵢⱼ^{D}, the dissipative force, and Fᵢⱼ^{R}, the random force as shown in Eq. (S4):

$${f}_{i} = {\sum }_{j \neq i} ( {F}_{i j}^{C} + {F}_{i j}^{D} + {F}_{i j}^{R} ) \qquad ( S4 )$$

All these forces act between beads i and j within a cutoff radius r_{c}, below which the interactions are neglected. The formula of conservative force is as follows:

$${F}_{i j}^{c} = \{ \begin{matrix}{a}_{i j} ( 1 - {r}_{i j} / {r}_{c} ) {\vec{e}}_{i j} , & {r}_{ij} < {r}_{c} \\ 0 , & {r}_{ij} > {r}_{c}\end{matrix} \qquad ( S5 )$$

where rᵢⱼ, aᵢⱼ, and \vec{e}ᵢⱼ are the distance between bead i and bead j, the repulsive parameter between different types of beads, and the unit vector from bead j to bead i.

The formulas of dissipative force Fᵢⱼ^{D} and random force Fᵢⱼ^{R} can be seen in Eqs. (S6) and (S7):

Fᵢⱼ^{D}=−γω²(rᵢⱼ)(\vec{e}ᵢⱼ·\vec{v}ᵢⱼ)\vec{e}ᵢⱼ (S6)

Fᵢⱼ^{R}=σω(rᵢⱼ)α(dt)^{-1/2}\vec{e}ᵢⱼ (S7)

where \vec{v}ᵢⱼ is the velocity vector difference between bead i and bead j, γ reflects the viscosity of fluid, α is a Gaussian random number with zero mean and unit variance reflecting the characteristic of Brownian interaction, and dt is the DPD timestep size. The value of σ is equal to the square root of (2K_{b}Tγ), where K_{b} is the Boltzmann constant and T is the system temperature.

The weighting factor ω(rᵢⱼ) used in the dissipative force and the random force has the form shown in Eq. (S8):

ω(rᵢⱼ)=1−rᵢⱼ/r_{c} (S8)

The large-scale atomic/molecular massively parallel simulator (LAMMPS) developed by Plimpton^{S9} was utilized to perform the DPD simulation. The dimensionless units for the length, time, and mass used in DPD simulation and their corresponding physical values were found (not shown).

The schematic diagram of the current DPD simulation model was shown in FIG. 9C. All DPD beads were initially placed in the face-centered cubic arrangement and the cylindrical and spherical shapes for Si nanowires and EVs, respectively, were directly built according to their corresponding geometries used in the experiment. During the DPD simulation, the Si nanowire was fixed and the EVs were treated as rigid bodies with diameters of about 50 nm. Periodic boundary conditions (PBC) were applied to the x and y dimensions and the length of a Si nanowire is 10 μm with the axial direction along the z dimension. The 48 EVs were placed 2 μm above the Si nanowires. The diameter of the Si nanowire is ca. 100 nm and the spacing between the nearest Si nanowires is 150 nm. The EV and Si nanowire beads are marked in blue and orange, respectively, in FIG. 9C. During the simulation, a timestep size of 0.005 was used for the trajectory integration for the first 100,000 steps. Then, a weak bias force along the −z direction was applied to the EVs to accelerate them toward the bottoms of Si nanowires. Note that the system maintained thermodynamic equilibrium for the first 100,000 steps and afterwards, a weak bias force was applied.

Several DPD parameters (aᵢⱼ=25.0, γ=67.5, r_{c}=1.7, k_{B}T=1.0, number density=3.0) used in the current study are the same as those in Gao's study,^{S10} which describes fluid flow in the microchannels. For modeling the EV capture process, the repulsive parameter between EV beads and Si nanowire beads was 0. Moreover, the repulsive parameter between different EVs was 30 to prevent EVs from merging.

After EVs were captured on the Si nanowire, water beads were shaded to visualize all captured EVs. By calculating relative frequencies of EVs located at different depths from the top of Si nanowires, frequency distribution histogram in FIG. 9D) shows that 52.1%, 25.0%, 14.6%, 8.3%, and 0% of EVs (n=48) were located at depths of 0-1 μm, 1-2 μm, 2-5 μm, 5-9 μm, and 9-10 μm from the top of Si nanowires, respectively. The fitting curve was obtained by using Eq. (S1) to describe the EV distribution probability profiles along the depth of Si nanowire from the DPD simulation. According to the curve-fitting formulas, y=43.83×exp(−0.954x), the T value was calculated as 1.048 μm, while the x₀ values are 43.83 for the DPD simulation. These results, derived from the experiment and DPD simulation, are in close agreement. It was seen that the EV distribution probability is reduced to its 0.368 (˜1/e) times x₀ value at depths lower than 1.5 μm.

FIGS. 9A-9E are images and graphs showing extracellular vesicle (EV) distribution probability profiles along the depth of Si nanowires analyzed by scanning electron microscopy (SEM) and computational simulation according to an embodiment. (FIG. 9A) A representative SEM image of EVs (diameter=30-300 nm) captured by Si nanowires (SiNWs). (FIG. 9B) Frequency distribution histogram of EVs (n=500, imaged by SEM) along the depth from the top of SiNWs and the exponential fit obtained empirically. (FIG. 9C) Schematic illustration of laminar boundary layer on the top of SiNWs. (FIG. 9D) The schematic diagram and result of dissipative particle dynamics (DPD) simulation model. EV and SiNW beads are marked in blue and orange, respectively. The diameters of each EV and SiNW are about 50 nm and 100 nm, respectively. The length of SiNW is 10 μm. The enlarged portions show the EV distribution along the depths of 0-1 μm, 1-2 μm, 2-5 μm, and 5-10 μm from the top of SiNW, respectively. (FIG. 9E) Frequency distribution histogram of EVs (n=48, simulated by DPD) along the depth from the tops of the SiNWs and the curve obtained by fitting an exponential function to the empirical data.

Extracellular-Vesicle-Capture Performance of NanoVilli Chips

FIGS. 10A and 10B are graphs showing extracellular-vesicle-capture performance of NanoVilli Chips according to an embodiment. (FIG. 10A) Flow rate effects on EV-capture performance using NanoVilli Chips (Si nanowire lengths=10-15 μm). (FIG. 10B) Extracellular vesicle distribution along the channel tested by segmentally quantifying RNA recovery rate of anti-EpCAM-conjugated SiNWS with only one channel and only two channels in comparison with the standard three channels (defined as 100%).

Quantification of Gene Alterations from Extracellular Vesicle-Derived RNA by Reverse Transcription Droplet Digital™ PCR

FIGS. 11A-11D are schemes illustrating reverse transcription Droplet Digital™ PCR (RT-ddPCR) analysis of gene alterations from extracellular vesicle (EV)-derived RNA according to an embodiment. (FIG. 11A) Schematic illustration of the variants of SLC34A2-ROS1 rearrangements in the HCC78 cell line and CD74-ROS1 rearrangements in non-small cell lung cancer (NSCLC) patients. (FIG. 11B) Workflow for RT-ddPCR analysis of ROS1 rearrangements. (FIG. 11C) Schematic diagram of the EGFR T790M mutation and wild-type in H1975 cell line and NSCLC patients. (FIG. 11D) Workflow for RT-ddPCR analysis of EGFR T790M mutation and wild type.

TABLE 2
Raw data of the average RNA recovery rates for the optimization
of NanoVilli Chips.

| | | RNA | | | |
|---|---|---|---|---|---|
| | | recovery rate | | | |
| Test factor | | $( = \frac{R N {A}_{c a p - E V} - R N {A}_{i}}{R N {A}_{ori - EV}} )$ | RNA_{c,} (ng) | RNA_{b.} (ng) | RNA (ng) |
| Flow rate | 0.1 | 54% | 63.5 | 2.5 | 113.4 |
| (mL h⁻¹) | 0.2 | 60% | 72.0 | 4.3 | 113.4 |
| (SiNWs = | 0.5 | 54% | 63.0 | 2.0 | 113.4 |
| 1-2 μm) | 1.0 | 48% | 56.7 | 1.8 | 113.4 |
| | 2.0 | 41% | 47.6 | 1.0 | 113.4 |
| Top | Flat | 31% | 36.3 | 2.0 | 111.6 |
| Topog- | 1-2 | 60% | 72.0 | 4.3 | 113.4 |
| raphy | μm | | | | |
| (length of | 10-15 | 82% | 97.1 | 6.0 | 111.6 |
| SiNWs) | μm | | | | |
| Anti- | 0 | 35% | 42.3 | 6.0 | 103.4 |
| EpcAm | 1.0 | 39% | 46.9 | 6.4 | 103.4 |
| μg mL⁻¹) | 2.5 | 45% | 53.9 | 7.0 | 103.4 |
| | 5.0 | 82% | 97.1 | 6.0 | 111.6 |
| | 10.0 | 81% | 96.5 | 6.9 | 110.4 |
| Flow rate | 0.1 | 71% | 84.8 | 6.6 | 110.4 |
| (mL h⁻¹) | 0.2 | 82% | 97.1 | 6.0 | 111.6 |
| (SiNWs = | 0.5 | 66% | 79.8 | 6.5 | 110.4 |
| 1-2 μm) | 2.0 | 46% | 56.1 | 5.9 | 110.4 |

TABLE 3
Raw data of the gene copy numbers detected from artificial plasma
samples containing different cell line-derived EVs by different methods.

| | | | | T790M |
|---|---|---|---|---|
| | | ROS1 | T790M | wild |
| EVs | Methods | rearrangement | mutation | type |
| HCC78 | NanoVilli Chips | 610 ± 55 | N/A^{a} | N/A^{a} |
| | Immunomagnetic | 206 ± 12 | N/A^{a} | N/A^{a} |
| | beads | | | |
| | Ultracentrifugation | 165 ± 8 | N/A^{a} | N/A^{a} |
| | Direct lysis | 37 ± 19 | N/A^{a} | N/A^{a} |
| H1975 | NanoVilli Chips | N/A^{a} | 1010 ± | 792 ± |
| | | | 42 | 216 |
| | Direct lysis | N/A^{a} | 27 ± 17 | 17 ± 6 |

^{a}N/A: not available.

Isolation of Artificial Plasma Samples by Magnetic Beads

To compare the EV capture performance of NanoVilli Chips with magnetic beads, Dynabeads™ MyOne™ Streptavidin Cl (Thermo Fisher Scientific, USA) were incubated with biotinylated anti-EpCAM (5.0 μg mL⁻¹, Abcam, USA) and washed 3 times prior to capture. For each capture study, 50-μL anti-EpCAM-coated Dynabeads™ (˜5×10⁸ beads) were incubated with 100-μL artificial plasma sample containing HCC78-derived extracellular vesicles (EVs) at room temperature for 30 min. After washing 3 times via magnetic separation, the EVs captured on magnetic beads were lysed with 600-μL Trizol solution (Zymo Research, USA). The EV-derived RNA was purified using a Direct-zol™ RNA MicroPrep Kit (Zymo Research, USA). The purified RNA was then measured with a Qubit™ 3.0 Fluorometer measurement and RT-ddPCR.

Collection of Blood Plasma Samples from Non-Small Cell Lung Cancer Patients

Non-Small Cell Lung Cancer Patient Enrollment and Blood Samples

Collection. Six treatment naïve advanced ROS1-positive^{S12} non-small cell lung cancer (NSCLC) patients (stages III and IV) from October 2016 to June 2017 and six relapsed EGFR positive NSCLC patients (stages III and IV) who were previously treated with epidermal growth factor receptor-tyrosine kinase inhibitors (EGFR-TKIs) and developed acquired resistance with known EGFR T790M^{S13} mutations from January to June 2018 were enrolled. Patients who had uncontrolled infection or Mycobacterium tuberculosis, or other uncontrolled malignant tumors, or severe mental disease were disqualified. All 12 enrolled patients received tyrosine kinase inhibitor (TKI) treatment according to clinical guidelines and underwent follow-up imaging examinations every 2-3 months for evaluation of clinical responses according to Response Evaluation Criteria in Solid Tumors (RECIST 1.1). This study was approved by the Ethics Committee of Guangdong Provincial Hospital of Traditional Chinese Medicine TCM and a written informed consent for this study was provided by each patient. Each 2.0 mL peripheral venous blood sample was collected in a BD Vacutainer glass tube (BD Medical, Fisher Cat. #02-684-26) with acid citrate dextrose from six ROS1 positive NSCLC patients before their first-line therapy. For the EGFR positive NSCLC patients, blood samples were collected at the time EGFR T790M mutations were confirmed on the re-biopsied tumor tissues. Among the 12 enrolled patients, some of the patients' blood samples were collected serially. About 1 mL plasma was isolated by centrifugation and 200 μL plasma was then run through a NanoVilli Chip under the optimum conditions. For each patient, 200 μL plasma was used for CK immunofluorescent staining and another 200 μL plasma for downstream RT-ddPCR.

Pathology Evaluation on Non-Small Cell Lung Cancer Tissues. Pathological examinations, including: Hematoxylin and eosin (HE) staining, immunohistochemistry (IHC), EGFR mutation analysis, and ROS1 rearrangement analysis, of the tumor tissues obtained from the 12 enrolled patients were performed with conventional laboratory methods in the pathology department of Guangdong Provincial Hospital of TCM. All tissue slides were reviewed independently by two pathologists from Guangdong Provincial Hospital of TCM. The tissues were fixed in 10% neutral formalin for 24-48 h and embedded in paraffin. The HE staining was performed by following Clinical Laboratory Improvement Amendments (CLIA)-compliant methods and equipment. All reagents, including 10% neutral formalin, xylene, ethanol, and acetone, were purchased from BoJing Company, China. Serial 3-4 μm-thick tissue sections from formalin-fixed paraffin embedded (FFPE) blocks were cut and mounted on poly-L-lysine coated glass slides. Standard IHC staining on 3-4-μm-thick tissue sections were performed on Ventana Benchmark ULTRA Slide Stainer according to the standard protocol. The IHC diagnostic panels of P63, CK5/6, CK7, TTF-1, Napsin A, CD56, synaptophysin (SYN), and chromogranin A (CgA) were routinely performed on each case to help distinguish NSCLC (adenocarcinoma, squamous cell carcinoma) from small cell lung cancer. Positive staining for CK7, Napsin A and/or TTF-1 combined with negative staining for P63, CK5/6, CD56, SYN, and CgA confirmed the diagnosis of NSCLC enrolled in the present study.

The EGFR mutations (including T790M mutation) were detected by the human EGFR gene mutation detection kit (YQ Biomed, Shanghai, China, China Food and Drug Administration, CFDA, approved) according to the manufacturer's instructions.^{S14} The ROS1 rearrangements were detected by reverse transcription (RT) using a fusion gene detection kit (Amoy, Xiamen, China, China Food and Drug Administration, CFDA, approved). Genomic DNA and Total RNA were extracted from FFPE tissue sections using Qiagen (Dusseldorf, Germany) QIAamp DNA FFPE Tissue Kit and RNeasy FFPE kit, respectively. Complement DNA was synthesized under the conditions 42° C., 1 h; 95° C., 5 min. Real-time PCR procedures were performed on a ViiA™ instrument (Life Technologies, Carlsbad, Calif., USA).

### Example 2

LINGO-1 Enables Specific Capture and Molecular Analysis of Ewing Sarcoma-Derived Extracellular Vesicles Via Bioorthogonal Ligation on Nanostructured Substrates

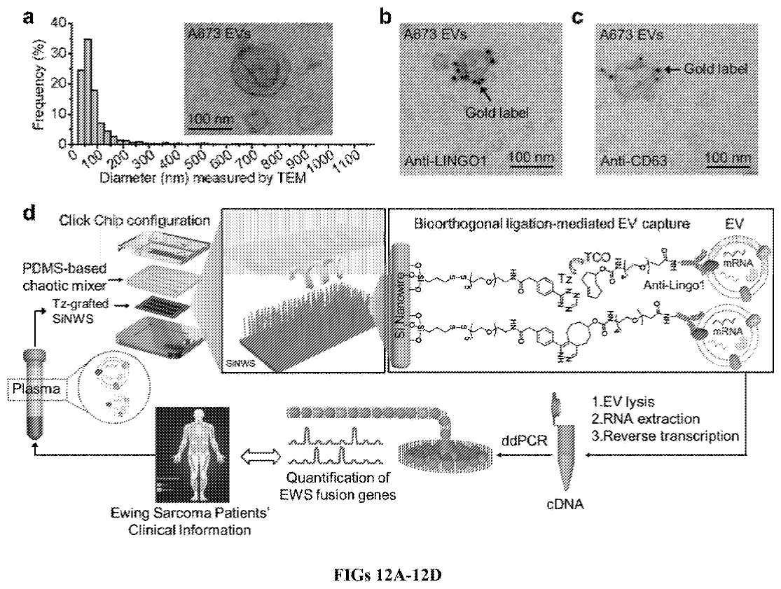

FIGS. 12A-12D are images, graphs and schematics showing that leucine-rich repeat and Ig domain protein 1 (LINGO1) enables specific capture of and molecular analysis of Ewing sarcoma (EWS)-derived extracellular vesicles (EVs) according to an embodiment. (FIG. 12A) Size distribution of A673 EWS cell-derived EVs measured by transmission electron microscopy (TEM). Inset: a representative TEM image of A673 EVs. (FIG. 12B) Immunogold-TEM of LINGO1 expression on A673 EVs. (FIG. 12C) Immunogold-TEM of CD63 expression on A673 EVs. (FIG. 12D) Scheme illustrating LINGO1 to recognize and capture EWS cell-derived EVs via the bioorthogonal Diels-Alder click reaction between trans-cyclooctene (TCO) and tetrazine (Tz) in silicon (Si) nanowire-embedded microfluidic chips (i.e. Click^{EV} chips), which is followed by in situ extraction of EV-derived mRNA for downstream analysis of EWS fusion genes via reverse transcription Droplet Digital™ PCR (RT-ddPCR).

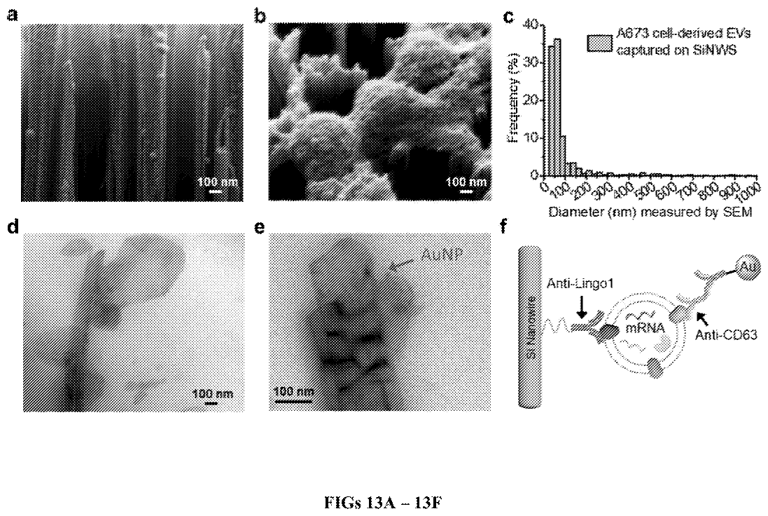

FIGS. 13A-13F are images, graphs and schematics showing the morphological characterization of A673 EVs captured via the reaction of TCO-anti-LINGO1 conjugates and Tz-grafted Si nanowires in Click^{EV} chips by electron microscopy according to an embodiment. A673 EVs are highlighted. (FIG. 13A) A scanning electron microscopy (SEM) image of A673 EVs attached on the sidewalls of Si nanowires. (FIG. 13B) A SEM image of A673 EVs immobilized on the tips of Si nanowires. (FIG. 13C) Size distribution of A673 EVs (n=621) captured on Si nanowires measured by SEM. (FIG. 13D) A transmission electron microscopy (TEM) image of A673 EVs immobilized on a Si nanowire. (FIG. 13E) Immunogold-TEM of A673 EVs labeled by anti-CD63 to the identity of EVs captured on Si nanowires. (FIG. 13F) Schematic illustrating the immunogold staining by mouse anti-CD63 and anti-mouse 10-nm gold on a EWS cell-derived EV captured on the sidewall of a Si nanowire via anti-LINGO1.

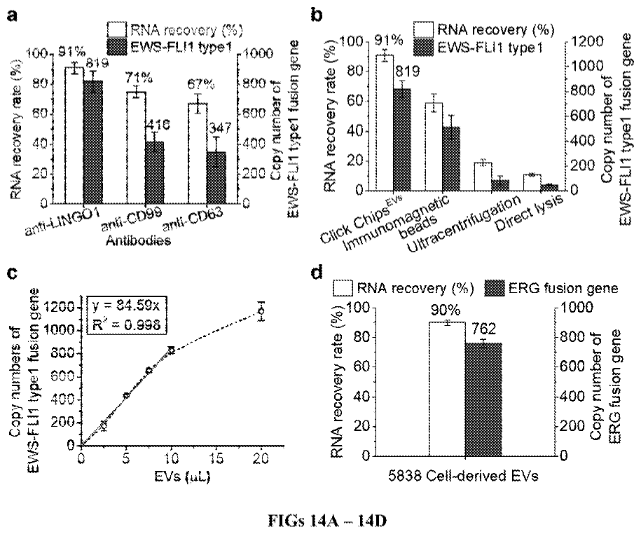

FIGS. 14A-14D are graphs showing validation and optimization of Click^{EV} Chips for LINGO1 induced capture of Ewing sarcoma (EWS) cell-derived EVs followed by quantification of EV-derived RNA according to an embodiment. (FIG. 14A) Comparison of RNA recovery rates and copy numbers of EWS-FLI1 type 1 fusion gene of A673 EVs enriched from artificial plasma samples by TCO-conjugated antibodies to LINGO1, CD99, and CD63 in Click^{EV} Chips, respectively. The final concentration of antibodies was 10 nM. (FIG. 14B) RNA recovery rates and copy numbers of EWS-FLI1 type 1 fusion gene observed for TCO-anti-LINGO1 induced A673 EV capture in Click^{EV} Chips and immunomagnetic beads, ultracentrifugation and direct lysis (as a control) using artificial plasma samples. (FIG. 14C) Dynamic ranges observed for quantification of EWS-FLI1 type 1 gene fusion from A673 EVs captured in Click^{EV} Chips. (FIG. 14D) General applicability of Click^{EV} Chips for EWS cell-derived EV capture followed by RNA quantification was validated using artificial plasma samples containing 5838 EVs harboring EWS-ERG fusion gene.

### Example 3

EpCAM and PSMA Enables Specific Capture and Molecular Analysis of Prostate Cancer-Derived Extracellular Vesicles

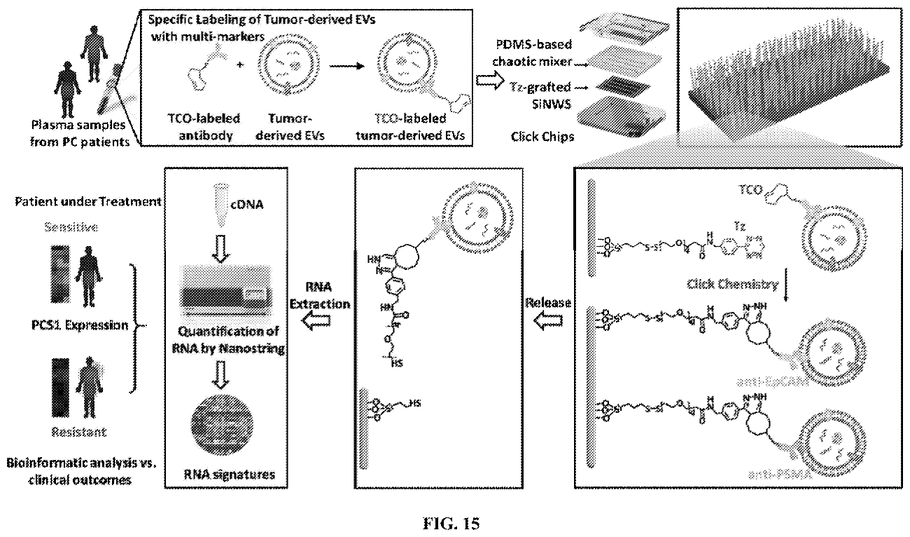

FIG. 15 is a scheme illustrating a nanostructured Click chip for specific recovery of tumor-derived EVs via multi-markers according to an embodiment. Tumor-derived EVs in blood plasma are targeted by TCO-labeled antibodies (anti-EpCAM and anti-PSMA), followed by a rapid capture by click chemistry onto the tetrazine modified silica nanowires. The captured EVs can be released specifically via a disulfide cleavage-driven by 1,4 dithiothreitol (DTT). The isolated EVs are then lysed to release EV-derived RNA, and then the quantification of disease-specific RNA is done using the NanoString nCounter® platform. Differential expression analysis of PC-specific RNA markers in a PC-specific panel will be performed for disease profiling.

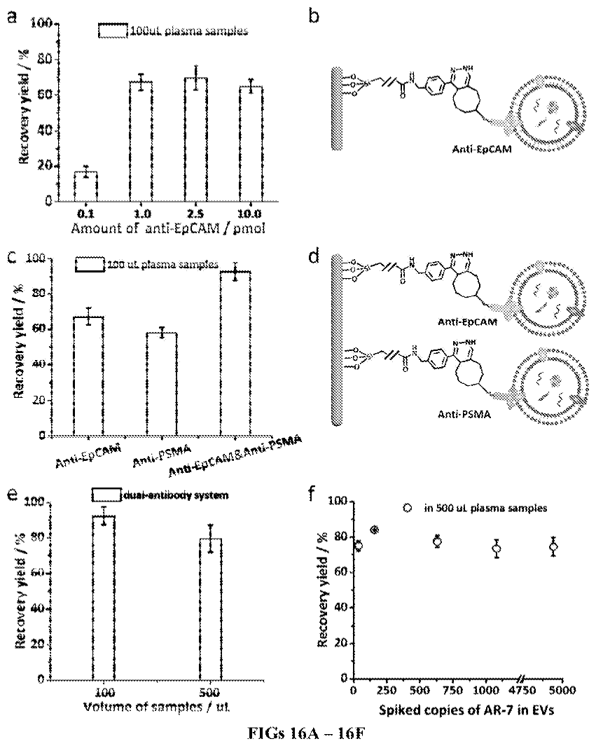

FIGS. 16A-16F are graphs and schematics showing validation and optimization of Click Chips using artificial plasma samples spiked with 22RV1 cell-derived EVs according to an embodiment. FIG. 16A) capture efficiency of 22RV1-derived EVs using a single capture agent was studied for Click Chips. FIG. 16B) Schematic illustrating the anti-EpCAM-mediated EV capture of Click Chips. FIG. 16C) Comparison of capture efficiencies for Click Chips via different markers. FIG. 16D) Schematic illustrating the multi-marker-mediated EV capture of Click Chips. e) Comparison of capture efficiencies for Click Chips processing plasma samples of 100 uL and 500 uL. f) Dynamic ranges of Click Chips for EV capture was validated using artificial samples with different concentration of 22RV1-derived EVs in 500 uL plasma.

### Example 4

Multi-Marker Cocktail Enables Specific Capture and Molecular Analysis of Hepatocellular Carcinoma-Derived Extracellular Vesicles

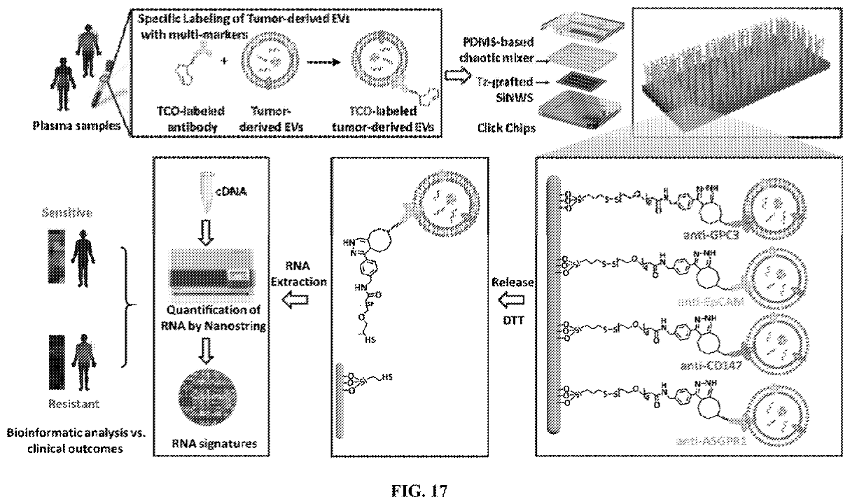

FIG. 17 is a scheme illustrating a nanostructured Click chip for specific recovery of HCC-derived EVs via multi-markers according to an embodiment. Tumor-derived EVs in blood plasma are targeted by TCO-labeled antibodies (anti-GPC3, anti-EpCAM, anti-CD147 and anti-ASGPR1), followed by a rapid capture by click chemistry onto the tetrazine modified silica nanowires. The captured EVs can be released specifically via a disulfide cleavage-driven by 1,4 dithiothreitol (DTT). The isolated EVs are then lysed to release EV-derived RNA, and then the quantification of disease-specific RNA is done using the NanoString nCounter® platform. Differential expression analysis of HCC-specific RNA markers in a HCC-specific panel will be performed for disease profiling.

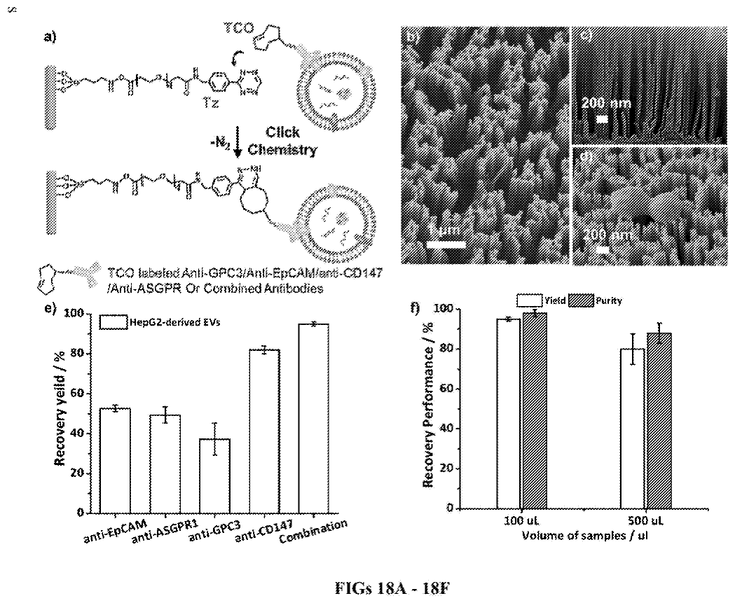

FIGS. 18A-18F are images and graphs showing validation and optimization of Click Chips using artificial plasma samples spiked with HepG2 cell-derived EVs according to an embodiment. FIG. 18A) Schematic illustrating the Click Chemistry-mediated EV capture after multi-marker recognition. SEM images of bare nanowires FIG. 18B), small EVs captured on nanowires FIG. 18C), and large EVs captured on the top of nanowires FIG. 18D). FIG. 18E) Comparison of capture efficiencies for Click Chips modified with different capture agents. FIG. 18F) Comparison of capture efficiency as well as capture purity for Click Chips processing plasma samples of 100 uL and 500 uL.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

### REFERENCES

(1) Shah, R.; Patel, T.; Freedman, J. E. Circulating Extracellular Vesicles in Human Disease. N. Engl. J. Med. 2018, 379, 958-966.
(2) Raposo, G.; Stoorvogel, W. Extracellular Vesicles: Exosomes, Microvesicles, and Friends. J. Cell Biol. 2013, 200, 373-383.
(3) Revenfeld, A. L.; Baek, R.; Nielsen, M. H.; Stensballe, A.; Varming, K.; Jorgensen, M. Diagnostic and Prognostic Potential of Extracellular Vesicles in Peripheral Blood. Clin. Ther. 2014, 36, 830-846.
(4) Garcia-Romero, N.; Esteban-Rubio, S.; Rackov, G.; Carrion-Navarro, J.; Belda-Iniesta, C.; Ayuso-Sacido, A. Extracellular Vesicles Compartment in Liquid Biopsies: Clinical Aplication. Mol. Aspects Med. 2018, 60, 27-37.
(5) Cocucci, E.; Meldolesi, J. Ectosomes and Exosomes: Shedding the Confusion between Extracellular Vesicles. Trends Cell Biol. 2015, 25, 364-372.
(6) Corso, G.; Mager, I.; Lee, Y.; Gorgens, A.; Bultema, J.; Giebel, B.; Wood, M. J. A.; Nordin, J. Z.; Andaloussi, S. E. Reproducible and Scalable Purification of Extracellular Vesicles Using Combined Bind-Elute and Size Exclusion Chromatography. Sci. Rep. 2017, 7, 11561.
(7) Akers, J. C.; Gonda, D.; Kim, R.; Carter, B. S.; Chen, C. C. Biogenesis of Extracellular Vesicles (EV): Exosomes, Microvesicles, Retrovirus-Like Vesicles, and Apoptotic Bodies. J. Neurooncol. 2013, 113, 1-11.
(8) Balaj, L.; Lessard, R.; Dai, L.; Cho, Y.-J.; Pomeroy, S. L.; Breakefield, X. O.; Skog, J. Tumour Microvesicles Contain Retrotransposon Elements and Amplified Oncogene Sequences. Nat. Commun. 2011, 2, 180.
(9) Eldh, M.; Bagge, R. O.; Lasser, C.; Svanvik, J.; Sjöstrand, M.; Mattsson, J.; Lindner, P.; Choi, D.-S.; Gho, Y. S.; Lötvall, J. Microrna in Exosomes Isolated Directly from the Liver Circulation in Patients with Metastatic Uveal Melanoma. BMC Cancer 2014, 14, 962.
(10) King, H. W.; Michael, M. Z.; Gleadle, J. M. Hypoxic Enhancement of Exosome Release by Breast Cancer Cells. BMC Cancer 2012, 12, 421.
(11) Gercel-Taylor, C.; Atay, S.; Tullis, R. H.; Kesimer, M.; Taylor, D. D. Nanoparticle Analysis of Circulating Cell-Derived Vesicles in Ovarian Cancer Patients. Anal. Biochem. 2012, 428, 44-53.
(12) Taylor, D. D.; Gercel-Taylor, C. The Origin, Function, and Diagnostic Potential of RNA within Extracellular Vesicles Present in Human Biological Fluids. Front. Genet. 2013, 4, 142.
(13) Melo, S. A.; Luecke, L. B.; Kahlert, C.; Fernandez, A. F.; Gammon, S. T.; Kaye, J.; LeBleu, V. S.; Mittendorf, E. A.; Weitz, J.; Rahbari, N.; Reissfelder, C.; Pilarsky, C.; Fraga, M. F.; Piwnica-Worms, D.; Kalluri, R. Glypican-1 Identifies Cancer Exosomes and Detects Early Pancreatic Cancer. Nature 2015, 523, 177-182.
(14) Reategui, E.; van der Vos, K. E.; Lai, C. P.; Zeinali, M.; Atai, N. A.; Aldikacti, B.; Floyd, F. P., Jr.; A, H. K.; Thapar, V.; Hochberg, F. H.; Sequist, L. V; Nahed, B. V.; B, S. C.; Toner, M.; Balaj, L.; D, T. T.; Breakefield, X. O.; Stott, S. L. Engineered Nanointerfaces for Microfluidic Isolation and Molecular Profiling of Tumor-Specific Extracellular Vesicles. Nat. Commun. 2018, 9, 175.
(15) Mohrmann, L.; Huang, H. J.; Hong, D. S.; Tsimberidou, A. M.; Fu, S.; Piha-Paul, S. A.; Subbiah, V.; Karp, D. D.; Naing, A.; Krug, A.; Enderle, D.; Priewasser, T.; Noerholm, M.; Eitan, E.; Coticchia, C.; Stoll, G.; Jordan, L. M.; Eng, C.; Kopetz, E. S.; Skog, J.; Meric-Bernstam, F.; Janku, F. Liquid Biopsies Using Plasma Exosomal Nucleic Acids and Plasma Cell-Free DNA Compared with Clinical Outcomes of Patients with Advanced Cancers. Clin. Cancer Res. 2018, 24, 181-188.
(16) Castellanos-Rizaldos, E.; Grimm, D. G.; Tadigotla, V.; Hurley, J.; Healy, J.; Neal, P. L.; Sher, M.; Venkatesan, R.; Karlovich, C.; Raponi, M.; Krug, A.; Noerholm, M.; Tannous, J.; Tannous, B. A.; Raez, L. E.; Skog, J. K. Exosome-Based Detection of EGFR T790M in Plasma from Non-Small Cell Lung Cancer Patients. Clin. Cancer Res. 2018, 24, 2944-2950.
(17) Tauro, B. J.; Greening, D. W.; Mathias, R. A.; Ji, H.; Mathivanan, S.; Scott, A. M.; Simpson, R. J. Comparison of Ultracentrifugation, Density Gradient Separation, and Immunoaffinity Capture Methods for Isolating Human Colon Cancer Cell Line LIM1863-Derived Exosomes. Methods 2012, 56, 293-304.
(18) Kalra, H.; Adda, C. G.; Liem, M.; Ang, C. S.; Mechler, A.; Simpson, R. J.; Hulett, M. D.; Mathivanan, S. Comparative Proteomics Evaluation of Plasma Exosome Isolation Techniques and Assessment of the Stability of Exosomes in Normal Human Blood Plasma. Proteomics 2013, 13, 3354-3364.
(19) Lamparski, H. G.; Metha-Damani, A.; Yao, J. Y; Patel, S.; Hsu, D. H.; Ruegg, C.; Le Pecq, J. B. Production and Characterization of Clinical Grade Exosomes Derived from Dendritic Cells. J. Immunol. Methods 2002, 270, 211-226.
(20) Caradec, J.; Kharmate, G.; Hosseini-Beheshti, E.; Adomat, H.; Gleave, M.; Guns, E. Reproducibility and Efficiency of Serum-Derived Exosome Extraction Methods. Clin. Biochem. 2014, 47, 1286-1292.
(21) Heinemann, M. L.; Ilmer, M.; Silva, L. P.; Hawke, D. H.; Recio, A.; Vorontsova, M. A.; Alt, E.; Vykoukal, J. Benchtop Isolation and Characterization of Functional Exosomes by Sequential Filtration. J. Chromatogr. A 2014, 1371, 125-135.
(22) Heinemann, M. L.; Vykoukal, J., Sequential Filtration: A Gentle Method for the Isolation of Functional Extracellular Vesicles. In Extracellular Vesicles: Methods and Protocols, Kuo, W. P.; Jia, S., Eds. Humana Press Inc: Totowa, 2017; Vol. 1660, pp 33-41.
(23) Rekker, K.; Saare, M.; Roost, A. M.; Kubo, A. L.; Zarovni, N.; Chiesi, A.; Salumets, A.; Peters, M. Comparison of Serum Exosome Isolation Methods for Microrna Profiling. Clin. Biochem. 2014, 47, 135-138.
(24) Davies, R. T.; Kim, J.; Jang, S. C.; Choi, E. J.; Gho, Y. S.; Park, J. Microfluidic Filtration System to Isolate Extracellular Vesicles from Blood. Lab Chip 2012, 12, 5202-5210.
(25) Kanwar, S. S.; Dunlay, C. J.; Simeone, D. M.; Nagrath, S. Microfluidic Device (Exochip) for On-Chip Isolation, Quantification and Characterization of Circulating Exosomes. Lab Chip 2014, 14, 1891-1900.
(26) Zhao, Z.; Yang, Y.; Zeng, Y.; He, M. A Microfluidic Exosearch Chip for Multiplexed Exosome Detection Towards Blood-Based Ovarian Cancer Diagnosis. Lab Chip 2016, 16, 489-496.
(27) Yang, F.; Liao, X.; Tian, Y.; Li, G. Exosome Separation Using Microfluidic Systems: Size-Based, Immunoaffinity-Based and Dynamic Methodologies. Biotechnol. J. 2017, 12, 1600699.
(28) Wang, Z.; Wu, H. J.; Fine, D.; Schmulen, J.; Hu, Y; Godin, B.; Zhang, J. X.; Liu, X. Ciliated Micropillars for the Microfluidic-Based Isolation of Nanoscale Lipid Vesicles. Lab Chip 2013, 13, 2879-2882.
(29) Wunsch, B. H.; Smith, J. T.; Gifford, S. M.; Wang, C.; Brink, M.; Bruce, R. L.; Austin, R. H.; Stolovitzky, G.; Astier, Y. Nanoscale Lateral Displacement Arrays for the Separation of Exosomes and Colloids Down to 20 nm. Nat. Nanotechnol. 2016, 11, 936-940.
(30) Lin, M.; Chen, J. F.; Lu, Y. T.; Zhang, Y.; Song, J.; Hou, S.; Ke, Z.; Tseng, H. R. Nanostructure Embedded Microchips for Detection, Isolation, and Characterization of Circulating Tumor Cells. Acc. Chem. Res. 2014, 47, 2941-2950.
(31) Chen, J. F.; Zhu, Y; Lu, Y. T.; Hodara, E.; Hou, S.; Agopian, V. G.; Tomlinson, J. S.; Posadas, E. M.; Tseng, H. R. Clinical Applications of Nanovelcro Rare-Cell Assays for Detection and Characterization of Circulating Tumor Cells. Theranostics 2016, 6, 1425-1439.
(32) Jan, Y. J.; Chen, J. F.; Zhu, Y.; Lu, Y T.; Chen, S. H.; Chung, H.; Smalley, M.; Huang, Y. W.; Dong, J.; Chen, L. C.; Yu, H. H.; Tomlinson, J. S.; Hou, S.; Agopian, V. G.; Posadas, E. M.; Tseng, H. R. Nanovelcro Rare-Cell Assays for Detection and Characterization of Circulating Tumor Cells. Adv. Drug. Deliv. Rev. 2018, 125, 78-93.
(33) Kohno, T.; Nakaoku, T.; Tsuta, K.; Tsuchihara, K.; Matsumoto, S.; Yoh, K.; Goto, K. Beyond ALK-RET, ROS1 and Other Oncogene Fusions in Lung Cancer. Transl. Lung Cancer Res. 2015, 4, 156-164.
(34) Rothschild, S. I. Targeted Therapies in Non-Small Cell Lung Cancer—Beyond EGFR and ALK. Cancers 2015, 7, 930-949.
(35) Maemondo, M.; Inoue, A.; Kobayashi, K.; Sugawara, S.; Oizumi, S.; Isobe, H.; Gemma, A.; Harada, M.; Yoshizawa, H.; Kinoshita, I. Gefitinib or Chemotherapy for Non-Small-Cell Lung Cancer with Mutated EGFR. N. Engl. J. Med. 2010, 362, 2380-2388.
(36) Crowley, E.; Di Nicolantonio, F.; Loupakis, F.; Bardelli, A. Liquid Biopsy: Monitoring Cancer-Genetics in the Blood. Nat. Rev. Clin. Oncol. 2013, 10, 472-484.
(37) Myung, J. H.; Park, S. J.; Wang, A. Z.; Hong, S. Integration of Biomimicry and Nanotechnology for Significantly Improved Detection of Circulating Tumor Cells (CTCs). Adv. Drug. Deliv. Rev. 2018, 125, 36-47.
(38) Peng, K. Q.; Yan, Y. J.; Gao, S. P.; Zhu, J. Synthesis of Large-Area Silicon Nanowire Arrays via Self-Assembling Nanoelectrochemistry. Adv. Mater. 2002, 14, 1164-1167.
(39) Lu, Y. T.; Zhao, L.; Shen, Q.; Garcia, M. A.; Wu, D.; Hou, S.; Song, M.; Xu, X.; Ouyang, W. H.; Ouyang, W. W.; Lichterman, J.; Luo, Z.; Xuan, X.; Huang, J.; Chung, L. W.; Rettig, M.; Tseng, H. R.; Shao, C.; Posadas, E. M. Nanovelcro Chip for CTC Enumeration in Prostate Cancer Patients. Methods 2013, 64, 144-152.
(40) Wang, S. T.; Liu, K.; Liu, J. A.; Yu, Z. T. F.; Xu, X. W.; Zhao, L. B.; Lee, T.; Lee, E. K.; Reiss, J.; Lee, Y. K.; Chung, L. W. K.; Huang, J. T.; Rettig, M.; Seligson, D.; Duraiswamy, K. N.; Shen, C. K. F.; Tseng, H. R. Highly Efficient Capture of Circulating Tumor Cells by Using Nanostructured Silicon Substrates with Integrated Chaotic Micromixers. Angew. Chem. Int. Ed. 2011, 50, 3084-3088.
(41) Sheng, W.; Ogunwobi, O. O.; Chen, T.; Zhang, J.; George, T. J.; Liu, C.; Fan, Z. H. Capture, Release and Culture of Circulating Tumor Cells from Pancreatic Cancer Patients Using an Enhanced Mixing Chip. Lab Chip 2014, 14, 89-98.
(42) Kármán, T. V. Über Laminare Und Turbulente Reibung. ZAMM 1921, 1, 233-252.
(43) Groot, R. D.; Warren, P. B. Dissipative Particle Dynamics: Bridging the Gap between Atomistic and Mesoscopic Simulation. J. Chem. Phys. 1997, 107, 4423-4435.
(44) Wang, S.; Cang, S.; Liu, D. Third-Generation Inhibitors Targeting EGFR T790M Mutation in Advanced Non-Small Cell Lung Cancer. J. Hematol. Oncol. 2016, 9, 34.
(45) Wu, W.; Haderk, F.; Bivona, T. G. Non-Canonical Thinking for Targeting ALK-Fusion Onco-Proteins in Lung Cancer. Cancers 2017, 9, 164.
(S1) Peng, K. Q.; Yan, Y. J.; Gao, S. P.; Zhu, J. Synthesis of Large-Area Silicon Nanowire Arrays via Self-Assembling Nanoelectrochemistry. Adv. Mater. 2002, 14, 1164-1167.
(S2) Lu, Y. T.; Zhao, L.; Shen, Q.; Garcia, M. A.; Wu, D.; Hou, S.; Song, M.; Xu, X.; Ouyang, W. H.; Ouyang, W. W.; Lichterman, J.; Luo, Z.; Xuan, X.; Huang, J.; Chung, L. W.; Rettig, M.; Tseng, H. R.; Shao, C.; Posadas, E. M. Nanovelcro Chip for CTC Enumeration in Prostate Cancer Patients. Methods 2013, 64, 144-152.
(S3) Wang, S.; Liu, K.; Liu, J.; Yu, Z. T. F.; Xu, X.; Zhao, L.; Lee, T.; Lee, E. K.; Reiss, J.; Lee, Y. K. Highly Efficient Capture of Circulating Tumor Cells by Using Nanostructured Silicon Substrates with Integrated Chaotic Micromixers. Angew. Chem. Int. Ed. 2011, 50, 3084-3088.
(S4) Peng, K. Q.; Yan, Y. J.; Gao, S. P.; Zhu, J. Synthesis of Large-Area Silicon Nanowire Arrays Via Self-Assembling Nanoelectrochemistry. Adv. Mater. 2002, 14, 1164-1167.
(S5) Wang, S.; Wang, H.; Jiao, J.; Chen, K. J.; Owens, G. E.; Kamei, K. i.; Sun, J.; Sherman, D. J.; Behrenbruch, C. P.; Wu, H. Three-Dimensional Nanostructured Substrates toward Efficient Capture of Circulating Tumor Cells. Angew. Chem. Int. Ed. 2009, 121, 9132-9135.
(S6) Sheng, W.; Ogunwobi, O. O.; Chen, T.; Zhang, J.; George, T. J.; Liu, C.; Fan, Z. H. Capture, Release and Culture of Circulating Tumor Cells from Pancreatic Cancer Patients Using an Enhanced Mixing Chip. Lab Chip 2014, 14, 89-98.
(S7) Karman, T. V Uber Laminare and Turbulente Reibung. ZAMM 1921, 1, 233-252.
(S8) Groot, R. D.; Warren, P. B. Dissipative Particle Dynamics: Bridging the Gap between Atomistic and Mesoscopic Simulation. J. Chem. Phys. 1997, 107, 4423-4435.
(S9) Plimpton, S. Fast Parallel Algorithms for Short-Range Molecular Dynamics. J. Comput. Phys. 1995, 117, 1-19.
(S10) Gao, C.; Zhang, P.; Marom, G.; Deng, Y.; Bluestein, D. Reducing the Effects of Compressibility in DPD-Based Blood Flow Simulations through Severe Stenotic Microchannels. J. Comput. Phys. 2017, 335, 812-827.
(S11) Stukowski, A. Visualization and Analysis of Atomistic Simulation Data with Ovito—The Open Visualization Tool. Model. Simul. Mater. Sc. 2009, 18, 015012.
(S12) Gainor, J. F.; Tseng, D.; Yoda, S.; Dagogo-Jack, I.; Friboulet, L.; Lin, J. J.; Hubbeling, H. G.; Dardaei, L.; Farago, A. F.; Schultz, K. R.; Ferris, L. A.; Piotrowska, Z.; Hardwick, J.; Huang, D.; Mino-Kenudson, M.; Iafrate, A. J.; Hata, A. N.; Yeap, B. Y.; Shaw, A. T. Patterns of Metastatic Spread and Mechanisms of Resistance to Crizotinib in ROS1-Positive Non-Small-Cell Lung Cancer. JCO Precis. Oncol. 2017, 2017.
(S13) Yang, J. C.; Ahn, M. J.; Kim, D. W.; Ramalingam, S. S.; Sequist, L. V.; Su, W. C.; Kim, S. W.; Kim, J. H.; Planchard, D.; Felip, E.; Blackhall, F.; Haggstrom, D.; Yoh, K.; Novello, S.; Gold, K.; Hirashima, T.; Lin, C. C.; Mann, H.; Cantarini, M.; Ghiorghiu, S.; Janne, P. A. Osimertinib in Pretreated T790M-Positive Advanced Non-Small-Cell Lung Cancer: Aura Study Phase II Extension Component. J. Clin. Oncol. 2017, 35, 1288-1296.
(S14) Xu, Q.; Zhu, Y.; Bai, Y.; Wei, X.; Zheng, X.; Mao, M.; Zheng, G. Detection of Epidermal Growth Factor Receptor Mutation in Lung Cancer by Droplet Digital Polymerase Chain Reaction. Onco. Targets Ther. 2015, 8, 1533-1541.

We claim:

1. A method for capturing extracellular vesicles from a fluid sample comprising:
providing a microfluidic chip, the microfluidic chip comprising:
a device for capturing extracellular vesicles from a fluid sample comprising:
a substrate; and
a plurality of nanowires at least one of attached to or integral with a surface of said substrate such that each nanowire of said plurality of nanowires has an unattached end; and
a membrane disposed on the device for capturing extracellular vesicles, the membrane comprising a fluid channel defined by a channel-defining layer;
wherein in the membrane is removable from the device for capturing extracellular vesicles,
wherein the plurality of nanowires comprise a binding agent attached to a surface region of the plurality of nanowires, and
wherein the channel-defining layer defines the fluid channel such that at least a portion of the fluid channel has a chaotic mixing structure to cause at least partially turbulent flow;
flowing the fluid sample through the fluid channel defined by the channel-defining layer so as to capture extracellular vesicles from the fluid sample;
removing the membrane from the device for capturing extracellular vesicles after the providing the fluid sample; and
collecting the extracellular vesicles captured from the fluid sample.

2. The method of claim 1, wherein the binding agent comprises a plurality of antibodies, and wherein the plurality of antibodies bind to two or more distinct targets.

3. The method of claim 1, wherein each of the plurality of nanowires has a length between 3-15 micrometers.

4. The method of claim 1, wherein each of the plurality of nanowires has a length between 10-15 micrometers.

5. The method of claim 1, wherein the chaotic mixing structure is configured in a herringbone pattern.

6. A method for determining the presence of a cancer cell in a subject comprising:
providing a microfluidic chip for capturing extracellular vesicles from a fluid sample, the microfluidic chip comprising:
a device for capturing extracellular vesicles from the fluid sample comprising:
a substrate; and
a plurality of nanowires at least one of attached to or integral with a surface of said substrate such that each nanowire of said plurality of nanowires has an unattached end; and
a membrane disposed on the device for capturing extracellular vesicles, the membrane comprising a fluid channel defined by a channel-defining layer;
wherein in the membrane is removable from the device for capturing extracellular vesicles,
wherein the plurality of nanowires comprise a binding agent attached to a surface region of the plurality of nanowires, and
wherein the channel-defining layer defines the fluid channel such that at least a portion of the fluid channel has a chaotic mixing structure to cause at least partially turbulent flow;
flowing the fluid sample through the fluid channel defined by the channel-defining layer so as to capture extracellular vesicles from the fluid sample;
assaying the captured extracellular vesicles for a presence of a biomarker associated with the cancer cell.

7. The method of claim 6, further comprising obtaining the fluid sample from the subject.

8. The method of claim 6, wherein the binding agent comprises a plurality of antibodies, and wherein the plurality of antibodies bind to two or more distinct targets.

9. The method of claim 6, wherein each of the plurality of nanowires has a length between 3-15 micrometers.

10. The method of claim 6, wherein each of the plurality of nanowires has a length between 10-15 micrometers.

11. The method of claim 6, wherein the chaotic mixing structure is configured in a herringbone pattern.

12. The method of claim 6, wherein the biomarker is a protein or a nucleic acid sequence.

13. A kit for capturing extracellular vesicles from a fluid sample comprising:
a microfluidic system for capturing extracellular vesicles from a fluid sample comprising:
a device for capturing extracellular vesicles from a fluid sample comprising:
a substrate; and
a plurality of nanowires at least one of attached to or integral with a surface of said substrate such that each nanowire of said plurality of nanowires has an unattached end; and
a membrane disposed on the device for capturing extracellular vesicles, the membrane comprising a fluid channel defined by a channel-defining layer;
a binding agent attached to a surface region of the plurality of nanowires; and
reagents for assaying the captured extracellular vesicles for a presence of a biomarker,
wherein in the membrane is removable from the device for capturing extracellular vesicles,
wherein the channel-defining layer defines the fluid channel such that at least a portion of the fluid channel has a chaotic mixing structure to cause at least partially turbulent flow.

14. The kit of claim 13, wherein the binding agent comprises a plurality of antibodies, and wherein the plurality of antibodies bind to two or more distinct targets.

15. The kit of claim 13, wherein each of the plurality of nanowires has a length between 3-15 micrometers.

16. The kit of claim 13, wherein each of the plurality of nanowires has a length between 10-15 micrometers.

17. The kit of claim 13, wherein the chaotic mixing structure is configured in a herringbone pattern.
